(19) United States
(12) Patent Application Publication
(10) Pub. No.: US 2025/0367872 A1
(43) Pub. Date: Dec. 4, 2025

(54) MICROFLUIDICS DEVICES AND PRINTING METHODS THEREFOR

(71) Applicant: YISSUM RESEARCH DEVELOPMENT COMPANY OF THE HEBREW UNIVERSITY OF JERUSALEM LTD., Jerusalem (IL)

(72) Inventors: Ofra BENNY, Jerusalem (IL); Eliana STEINBERG, Jerusalem (IL)

(21) Appl. No.: 18/876,446

(22) PCT Filed: Jun. 22, 2023
(86) PCT No.: PCT/IL2023/050648

§ 371 Date: Dec. 18, 2024

Related U.S. Application Data
(60) Provisional application No. 63/366,794, filed on Jun. 22, 2022.

(51) Int. Cl. B29C 64/124; B01L 3/00; B29C 64/30; B29C 71/00; B29L 31/00; B29L 31/60; B33Y 10/00; B33Y 40/20; C12M 3/06

(52) U.S. Cl. CPC B29C 64/124; B01L 3/502707; B01L 3/502746; B29C 64/30; B29C 71/0009; B33Y 10/00; B33Y 40/20; C12M 23/16; B01L 2200/0694; B01L 2200/12; B01L 2300/042; B01L 2300/0864; B01L 2300/0867; B01L 2300/087; B01L 2300/0874; B01L 2300/0883; B29C 2071/0027; B29L 2031/60; B29L 2031/756

(57) ABSTRACT

The disclosure provides microfluidic chips and systems for maintaining viability of biological sample, and methods for their production by direct 3-D printing of biocompatible UV-curable polymeric resins.

### TECHNOLOGICAL FIELD

The present disclosure concerns microfluidic chips and systems, typically for maintaining viability of biological sample for the purpose of developing patient-specific tailored treatments, and methods for their production.

### RESEARCH FUNDING STATEMENT

The project leading to this application has received funding from the European Research Council (ERC) under the European Union's Horizon 2020 research and innovation program (grant agreement No 756762); Israel Cancer Association (ICA) No. 0394691; Israel Foundation of Science (ISF) No. 0394883; Israel Ministry of Science and Technology (MOST) No. 0394906; David R. Blum Center for Pharmacy at The Hebrew University; Adams Fellowship Program of the Israel Academy of Sciences and Humanities (ES).

### BACKGROUND ART

References considered to be relevant as background to the presently disclosed subject matter are listed below:
Goldstein et al., Micromachines 2021, 12, 627
Au et al., Lab Chip 2014, 14, 1294-1301
Sackmann et al., 2014, doi:10.1038/nature13118

Acknowledgement of the above references herein is not to be inferred as meaning that these are in any way relevant to the patentability of the presently disclosed subject matter.

### BACKGROUND

Microfluidic devices (also known as microfluidic chips) are devices used in various studies carried out on cells, cells clusters, or tissue samples, in which the cells are exposed to different controlled conditions to assess their behavior under such conditions. The chips typically include micro-channels that are connected together to allow fluids to pass therethrough in various desired flow patterns, to form a network of channels between inlet and outlet ports.

Microfluidic devices play an important role in numerous biological, chemical and engineering applications. For example, the organ-on-a-chip technology (OOAC) focuses on the biomimetic emulation of tissue characteristics in a microfluidic device, typically permitting spatial and temporal control over cellular microenvironment for the cultured cells.

By another example, it is well established that tumors display substantial intratumor, intertumor intrapatient and interpatient heterogeneity, making the “one-size-fits-all” conventional treatment approach not sufficiently effective in combating cancer. Even patients diagnosed with the same kind of cancer may present different tumor phenotypes and respond differently to the same treatment. Due to the many complexities of cancer, the development of reliable tumor tissue culture models that can mimic a range of malignancy behaviors more physiologically accurately would be of great value in the battle against cancer. Such models could also be clinically relevant as predictive drug-performance tools, enabling physicians to replace treatment selection through “trial and error” with rational selection of the most effective treatment for each patient.

Widely used two-dimensional (2D) cell cultures lack key features that are critical for recapitulating physiological systems, such as: spatial cell-cell interactions, extra-cellular matrices (ECM), dynamic metabolic demand, increased hypoxia due to mass growth, and effects of the tumor's microenvironment (TME). 2D-culture inaccuracies in cytotoxicity assays can lead to misinterpretation and poor prediction of in vivo behavior. For example, tissue processes such as hypoxia are known to contribute to treatment resistance. Drug screening in monolayer models, that have been the main drug selection tool for years, may be partly responsible for the high rate of clinical trial failures for new molecules.

To that end, 3D cellular models are being developed and studied extensively. One of the most promising 3D cellular models is the multicellular tumor spheroid model. Spheroids are ex vivo cellular aggregate “micro-tissues” that exhibit tissue-like metabolic activity that is governed by nutrient and oxygen diffusion mechanisms similar to tumors. These conditions are similar to hypoxic micro-tumors in vivo that are known to negatively affect a tumor's sensitivity to anticancer drugs and to contribute to its acquired resistance.

However, despite the many advantages of 3D cultures, the lack of flow results in their failure to capture the real complexity of tissues. For example, flow conditions subject tissues to mechanical forces generated by fluid shear stress, hydrostatic pressure and tissue deformation, that can substantially influence cancer cell behavior. Animal models recapitulate more closely the in vivo TME complexity, however they raise ethical concerns and are not always a good representation of the human pathophysiology. The shift towards human tissue models of high physiological mimicry substantially reduces the high costs that are associated with the use of animals and solves many of the ethical issues. In this regard, organ-on-a-chip platforms open important possibilities that can become the future state of the art, especially in light of the Food and Drug Administration (FDA) recent announcement regarding the acceptance of animal alternatives in the track of drug approvals.

Meeting these needs, tumor-on-chip technologies have the prospect of enabling the recapitulation of the physiologically relevant physical microenvironment of cancers while sustaining fluid perfusion in vitro. The standard approach used to fabricate microfluidic devices is based on casting techniques using mainly polydimethylsiloxane (PDMS) (Au et al. 2014; Sackmann et al. 2014). This is owed to PDMS's favorable properties such as biocompatibility, optical transparency, and gas permeability. However, its tendency to bind or adsorb small hydrophobic molecules make it less suitable for drug-based studies, since it may change target concentrations and result in drug delivery to undesired regions in the microfluidic device. In addition, PDMS's lack of durability for lengthy experiments, the requirement of a clean room setup for fabrications, and extensive manual procedures has resulted in the increasing use of stereolithography (SLA) 3D printing as an alternative fabrication method for microfluidic devices. In this fabrication method, 3D structures are built in a layer-by-layer photopolymerization deposition with a UV-curable liquid resin.

Printing in 3D with photocurable resins enables versatility and complexity in device designs that are often impossible or very difficult to obtain otherwise. Moreover, 3D printing may considerably reduce the post-processing time and costs. Despite the obvious benefits, a substantial obstacle standing in the way of applying 3D printing for cell culture studies is that SLA resins are not sufficiently biocompatible and their limited optical transparency limits sample visualization. Most of the commercially available photocurable resins have proprietary formulations with scarce information regarding their composition and cytocompatibility.

### GENERAL DESCRIPTION

The present disclosure provides methods for manufacturing microfluidic devices for maintaining live biological samples in a microfluidic system by direct printing of a printable biocompatible resin on a hydrophilic substrate. The disclosure further provides various designs of the device to obtain different flow regimens, thereby permitting strict control over the microenvironment to which test cells or tissues are exposed.

By their unique design, the devices of this disclosure permit maintaining biological samples, e.g. patient-derived multicellular spheroids, for long periods of time, for example, for permitting drug screening tests for personalized therapy purposes. Further, the direct printing method utilized to produce devices of this disclosure permit limitless flexibility of the design and architecture of the devices, including various shapes of channels or features in the z-axis, thereby tailoring the required flow conditions for each biological sample, forming gradients of a desired agents, and/or enabling subjecting cells to different ratios of two or more agents.

As will be explained further below, unlike PDMS devices used to date which are permanently sealed and do not provide access to the biological sample, the disclosure further provides a microfluidic device that is designed for multiple-use, permitting multiple opening and closing actions by a unique capping design that provide a tight seal which can withstand the pressure created during the microfluidic flow.

According to one of its aspects, the disclosure provides a method for obtaining a microfluidic device suitable for maintaining a biological sample in viable conditions, the method comprises:
(a) printing a first layer of a biocompatible UV-curable polymeric resin onto a substrate having a hydrophilic surface;
(b) applying UV radiation to cure said first layer;
(c) printing subsequent layers of said biocompatible UV-curable polymer resin onto the first layer, in a layer-by-layer mode according to pre-determined layer patterns, such that each of the subsequent layers having a thickness at least about an order of magnitude larger than that of the first layer, to form a microfluidics structure over said first layer;
(d) applying UV radiation to obtain a cured device;
(e) immersing said cured device in at least one solvent for a period of time sufficient to leach remainders of uncured biocompatible UV-curable polymeric resin from said cured device;
(f) removing said cured device from the solvent after said period of time to obtain said microfluidic device.

The method of this disclosure permits direct printing of UV-curable resins onto suitable substrates, i.e. a substrate having a hydrophilic surface, that enables obtaining sufficient adhesivity of the resin to the substrate. For this purpose, the substrate is a hydrophilic substrate, as will be explained below, that has a surface energy match to the UV-curable resin. Further, in order to ensure proper adhesivity, the first printed layer is significantly less thick, i.e. by at least an order of magnitude, compared to the ensuing printed layers, and is treated for complete curing before printing of ensuing layers. The combination of surface energy match and the low thickness of the first printed layer ensure good adherence of the device to the substrate, without the risk of peeling or detachment from the substrate.

The term microfluidic device (or microfluidic chip) is meant to denote a device having a plurality of channels or channels arrays, having micrometric diameter, through which small amounts of fluids can be introduced to tested samples in a controlled manner. The structure of the channels or the channels arrays can be tailored to obtain certain flow characteristics or defined concentrations of fluids to be introduced to the samples. The channels may have different inner diameters, typically ranging between about 5 μm (micrometers) and about 1200 μm, e.g. between about 5 μm and about 500 μm.

The fluid can be in the form of a liquid or a gas.

The microfluidic devices of this disclosure are designed to maintain a biological sample in viable conditions and expose the biological sample to a variety of environments, for example for assessing the effect of different environments on the samples. The term biological sample refers to any sample derived from an organism, for example single cells (eukaryotic or prokaryotic), cell clusters and aggregates and suspensions, spheroids, organelles, organoids, micro-organs tissue samples, tissue cultures, biopsies, tissue scaffolds, extracellular matrices (ECM), etc. Maintaining the biological sample viable means keeping the biological sample alive for a desired period of time, typically according to needs of the test design. The term also means to encompass enabling cells in the sample to grow, proliferate, differentiate or react to various agents that are provided to the sample while held in the device.

For this purpose, as explained above, the methods of this disclosure are aimed at providing devices that are biocompatible with the biological sample by a direct printing protocol. In methods of this disclosure, sufficient adhesion is first obtained onto a substrate by printing a thin layer of biocompatible UV-curable polymeric resin onto the substrate, the substrate having a hydrophilic surface.

According to some embodiments, the thickness of the first layer is at most about 0.05 mm (millimeters). According to other embodiments, the thickness of the first layer is at most about 0.04 mm. According to some other embodiments, the thickness of the first layer is at most about 0.03 mm. According to yet other embodiments, the thickness of the first layer is at most about 0.02 mm.

The term UV-curable polymeric resin refers to a resin in liquid form, that comprises one or more monomers and a UV-sensitive curing agent and/or UV-sensitive curing initiator. Once exposed to radiation in the ultraviolet range and a proper wavelength, the curing initiator or curing agent initiates a chemical polymerization reaction of the monomers, to form long polymeric chains.

The term biocompatible refers to a resin that is essentially non-toxic or lacking injurious impact on the biological sample which the cured resin comes in contact with, thereby having no substantive effect on the viability of the biological sample. Further, the biocompatible resin has essentially no adverse impact on the growth and any other desired characteristics of the biological sample coming into contact with the cured resin.

The biocompatible UV-curable polymeric resins are stereolithography (SLA) and digital light processing (DLP) resins that comprise UV-curable moieties, typically multifunctional epoxy or (meth)acrylate monomers. For example, epoxy resins are cured in a step-growth manner in the presence of amines or anhydrides, whereas acrylate monomers generally undergo radical chain-growth polymerization.

According to some embodiments, the biocompatible UV-curable resin is selected as to obtain transparency once polymerized. The term transparency (or transparent or any lingual variation thereof) means transparency to light in the visible spectrum. The transparency of the devices of this disclosure is utilized to enable analysis by visual means (e.g. microscopy) of the biological samples within the device.

By preferred embodiments, the resin is hydrophilic. The first layer, as noted, is hence directly printed onto a substrate that has a hydrophilic surface. The term hydrophilic refers to a material, a molecule or a moiety that exhibits affinity for water. By matching the hydrophilicity, or polarity, of the surface of the substrate to that of the biocompatible UV-curable polymeric resin, good adhesion of the resin to the substrate after curing can be obtained. Further, by printing the first layer of the biocompatible UV-curable polymeric resin in a significantly reduced thickness compared to the ensuing layers, even distribution of stresses can be obtained in the first layer after curing, thereby increasing adhesiveness and minimizing detachment of the device from the substrate.

The substrate can be in any suitable shape or form, and can be rigid or pliable. According to some embodiments, the substrate is transparent to permit analysis of the sample(s) held in the device.

According to some embodiments, the substrate is made of a hydrophilic material, such as a hydrophilic polymer.

According to other embodiments, the substate is made of surface-treated plastic, for example a plastic substrate having a plasma-treated surface or coated by one or more hydrophilic materials.

According to some other embodiments, the substrate is made of glass, coated by one or more hydrophilic moieties. In such embodiments, the method can further comprise step (0), before step (a), step (0) comprises coating a glass surface by one or more hydrophilic moieties. For example, the glass surface can be coated with hydrophilic molecules such as 3-(trimethoxysilyl) propyl methacrylate (TMSPMA).

According to some embodiments, the first layer is substantially continuous over the hydrophilic surface. According to some embodiments, at least one of said subsequent layers is non-continuous to thereby form at least a portion of said microfluidics structure. In other words, while the first layer is typically continuous, at least one of the subsequent layers is non-continuous, to thereby define the microfluidic structure elements (such as fluid channels, sample chamber, inlet and outlet ports, etc.), while some other subsequent layers can be continuous.

The subsequent layers are printed layer-by-layer onto the first layer. In the context of the present disclosure, layer-by-layer printing means to denote printing of a layer of resin, followed by partial curing thereof (by UV irradiation, for example) before printing the next layer thereonto. Hence, UV-radiation is typically applied between printing of each subsequent layer to partially cure and stabilize said subsequent layer before printing the next layer thereonto.

In order to prevent unintentional curing of the biocompatible UV-curable polymeric resin during the process, steps (a) and (c) are carried out in dark conditions. Dark conditions refers to prevention of exposure to light in the UV wavelengths.

In case of methods in which step (0) is carried out, and the hydrophilic coating is UV-sensitive, step (0) is also carried out in dark conditions.

After printing is completed, UV-radiation is applied to complete the curing reaction of the resin, resulting in a cured device adhered to the substrate and having the desired structure according to the patterns of printing. Once cured, the device is then immersed in at least one solvent for a period of time that is sufficient to leach out remainders of uncured species or moieties (such as unreacted monomers or oligomers, initiators, curing agents, etc.).

The printed device can, by some embodiments, also be immersed in at least one solvent for an initial period of time to pre-remove at least part of the remainders of uncured species or moieties, the solvent may be the same or different from the solvent used in step (e).

The solvent is a liquid, in pure form or a mixture of liquids, in which the unreacted species are at least partially soluble, however the cured polymeric resin and the substrate are insoluble. Hence, by treating the cured device in said solvent, undesired impurities, such as the unreacted resin species, which are typically toxic to the biological sample, can be extracted out of the device. By some embodiments, said period of time is at least about 6 hours, e.g. between about 6 hour and about 48 hours. According to some embodiments, the period of time is between about 12 hours and about 36 hours.

According to some embodiments, the solvent is selected from C₂-C₆ alcohol, for example ethanol, isopropanol, butanol, pentanol, hexanol and mixtures thereof. According to some embodiments, the solvent is ethanol, isopropanol, or mixtures thereof.

According to some embodiments, the method further comprises washing the cured device with at least one solvent prior to step (f).

By another aspect, this disclosure provides a microfluidic device suitable for maintaining a biological sample in viable conditions obtained by the method described herein.

By yet another aspect, there is provided a microfluidic device suitable for maintaining a biological sample in viable conditions, the microfluidic device comprising:
at least one fluid inlet port and at least one fluid outlet port defining a fluid flow path therebetween;
at least one biological sample holding chamber, positioned between the at least one fluid inlet port and at least one fluid outlet port in said flow path;
an array of fluid feed channels, the channels linking between said at least one inlet port and said at least one biological sample holding chamber in said flow path; and
at least one fluid draining channel linking said at least one biological sample holding chamber to said at least one outlet port in said flow path;
said microfluidic device being obtained by the method disclosed herein.

Various configurations of the device can be provided, depending on the desired function of the device. One example is a microfluidic device designed to expose a singular biological sample to defined microenvironments. Another example is a microfluidic device designed to hold a plurality of identical or different biological samples, and expose each of the samples to one or more microenvironments. By yet another example, the microfluidic device is designed to receive two or more fluids, and mix these at different ratios, thus exposing the biological samples to different ratios of test agents in the microenvironment. In another example, the microfluidic device is designed to maintain one or more samples under hypoxic conditions during flow of one or more fluids to the samples.

The term microenvironment defines physical and/or chemical conditions to which the sample is exposed. The microenvironment includes chemical composition of the fluids, pressure, temperature, flow rate, flow regimen (e.g. laminal, turbulent), shear forces, etc., alone or in any combination.

By another one of its aspects (to be referred to as the “first device aspect”), the disclosure provides a microfluidic device suitable for maintaining one or more biological samples in viable conditions and exposing the biological samples to a plurality of different environments, the microfluidic device comprising:
n fluid inlet ports and at least m fluid outlet ports defining a generally axial direction from the inlet ports to the outlet ports along a plane of the device, the inlet ports and the outlet ports defining a fluid flow path therebetween;
m biological sample holding chambers, positioned between the fluid inlet ports and fluid outlet ports in said flow path;
an array of fluid feed channels linking between said inlet ports and said biological sample holding chambers in said flow path, the array comprises:
   a distribution manifold,
   at least one first set of at least m first channels, each first channel linking between the distribution manifold and a corresponding biological sample holding chamber, each of the first channels having at least a portion thereof defined in said plane and at least one other portion thereof vertically distanced from said plane; and
at plurality of fluid draining channels, corresponding to the number of biological sample holding chambers, each fluid draining channel linking between a biological sample holding chamber and a corresponding outlet port;
wherein n≥1, m is n+1.

The microfluidic devices of the first device aspect and the second device aspect (to be defined below) are typically designed to introduce one or more fluids into the device via fluid inlet ports, typically two or more fluids that differ in composition (for example depending on the desired parameter of the biological sample to be tested). The inlet ports are connected to biological sample holding chambers via an array of fluid feed channels, thereby permitting flow of fluids from the inlet ports to the chambers.

The device includes n fluid inlet ports, and m biological sample holding chambers, such that n≥1 and m is n+1. In other words, the number of chambers is larger than the number of inlet ports.

The array of fluid feed channels comprises a distribution manifold, and at least one set of first channels. The distribution manifold fluidly links between the inlet ports and the first channels, such that fluid received through the inlet ports is distributed to the first channels. When the device comprises two or more inlet ports, the distribution manifold also functions to partially mix the fluids fed through the inlet ports before introduction into the first channels.

Each of the biological sample holding chambers is fluidly linked to a corresponding outlet port via a respective fluid draining channel, as to permit draining of fluid from the chamber.

The inlet port(s) and the outlet port(s), therefore, define between them a fluid flow path that is designed to deliver fluids to the biological sample holding chamber to create desired microenvironments within the chambers.

The inlet port(s) and the outlet port(s) further define a generally axial direction therebetween along a main plane of the device. To increase the length of the flow path of fluids before introduction into the biological sample holding chamber(s), e.g. to obtain better mixing of two or more fluids, the first channels having at least a portion thereof defined in said main plane and at least one other portion thereof vertically distanced from said main plane (i.e. distanced along the z-direction of the device). In other words, the first channels can have several continuous portions, some of which are defined within the main plane of the device and the others in one or more planes of the device that are vertically distanced from the main plane. By modifying the number of portions that are within the main plane and out of the main plane, as well as the shape and diameters of the first channels, different mixing and flow regimens can be obtained in a relatively compact configuration of the device. The channels have an overall axial direction.

According to some embodiments, n≥2.

According to other embodiments, the number of biological sample holding chambers is at least m+1, and the array comprises:
at least one second set of at least m+1 second fluid feed channels, each of the second channels having at least a portion thereof defined in said plane and at least one other portion thereof vertically distanced from said plane, and each second channel being linked to a corresponding biological sample holding chamber; and
a collection channel, linking between said first set and said second set.

In such embodiments, the device comprises at least two sets of fluid feed channels: a first set and a second set. The configuration (e.g. shape or diameter) of the second channels can be the same or different from the first channels. The first and second sets are fluidly linked therebetween by a collection channel, such that fluids from the first channels are collected in the collection channel, and distributed from the collection channel to the second channels.

By another aspect (referred to herein as the “second device aspect”), there is provided a microfluidic device for maintaining one or more biological samples in viable conditions and exposing the biological samples to a plurality of different microenvironments, the microfluidic device comprising:
n fluid inlet ports and (n+i) fluid outlet ports defining a generally axial direction from the inlet ports to the outlet ports along a main plane of the device, the inlet ports and the outlet ports defining a fluid flow path therebetween;
(n+1) biological sample holding chambers, positioned between the fluid inlet ports and fluid outlet ports in said flow path;
an array of fluid feed channels linking between said inlet ports and said biological sample holding chambers in said flow path, the array comprises:
   a distribution manifold,
   p sets of fluid feed channels, the number of channels in each set p₍ᵢ₎ being n+i, each of fluid feed channels having at least a portion thereof defined in said main plane and at least one other portion thereof vertically distanced from said main plane; each first channel linking between the distribution manifold and a corresponding biological sample holding chamber;
   (i−1) collection channels, each collection channel linking between two adjacent sets of fluid feed channels; and
(n+i) fluid draining channels, each fluid draining channel linking between one of the biological sample holding chambers and a corresponding outlet port;
   wherein n is the number of inlet ports, n≥1,
   p is the number of sets of fluid feed channels, p≥1, and
   i is an integer index numeral counting the set of fluid feed channels, i≥1.

The embodiments below refer both to the first device aspect and to the second device aspect. Such devices can be utilized to form a gradient of concentrations of an agent, thereby exposing the biological sample to gradually decreasing concentration (or relative concentration) of one or more agents, typically an agent having one or more effects on the biological sample.

The agent can be any one of a therapeutic agent, a diagnostic agent, a contaminant, a toxin, a chemotherapeutic agent, a biological agent, a pH modifying agent, an agent required for viability and metabolism of the biological sample, an agent used in culturing, a chelating agent, a sustaining agent, a staining agent, a fixating/permeabilizing agent, enzymes, antibodies, growth factors, modulating agents, particles/beads, immunological agent, etc.

By some embodiments, the sets of channels are arranged in a staggered manner along the main plane. In other words, the channels of a given set are arranged off-set from the channels of an ensuing set located along the axial direction of the device. This allows for formation of concentration gradient or mixing ratios between fluids, each collecting channel receiving fluids from a previous channels set and distributing the fluids to the ensuing channels set, thereby forcing fluids introduced from a pervious set to be mixed in the ensuing set of channels. For example, in a device that comprises 2 inlet ports and 3 sets of channels: a first set with 3 channels, a second set with 4 channels and a third set with 5 channels. In the first set, a first channel will be fed 100% of the first fluid through the manifold, a second channel will be fed a 50%-50% of the first and second fluids, while the third channel will be fed 100% of the second fluid. The fluids from the first set are further mixed therebetween in the second set, and from there further mixed in the third set-eventually resulting in 5 different relative concentrations of the two fluids that are each fed to a corresponding biological sample holding chamber (100% first fluid, 25%+75%, 50%+50%, 75%+25% and 100% of the second fluid). In this manner, the structure of the device permits exposure of the biological samples to a variety of different environments in a compact and highly tailorable arrangement.

The channels (i.e. each of the fluid feed channels, the collection channels and/or the drain channels) may have different inner diameters, typically ranging between about 5 μm and about 1200 μm, typically between about 5 μm and about 500 μm.

By some embodiments, the collection channel(s) is (are) oriented substantially perpendicular to the axial direction and defined within the main plane. However,

By some other embodiments, the collection channel(s) is (are) substantially perpendicular to the axial direction and defined within a plane of the device that is vertically distanced from the main plane (i.e. distanced along the z-direction of the device). In some embodiments, where there are two or more collections channels, all channels can be on the same plain or some of the channels can be on different planes (e.g. at least one of the channels being on the main plane and at least one other of the channels on a plane vertically distances from the main plane).

According to some embodiments, the fluid feed channels in each set of channels are identical to one another. According to other embodiments, the fluid feed channels in each set of channels are different one from the other.

According to some embodiments, the fluid feed channels are the same in all of the sets of channels. According to some other embodiments, the fluid feed channels in at least one of the sets of channels are different from the channels in the other sets of channels.

By some embodiments, the fluid feeding channels are substantially linear along the axial direction.

By some other embodiments, the fluid feed channels are curved.

By yet other embodiments, the fluid feeding channels are spiral.

The curved, or preferably spiral channels, enable thorough mixing of the fluids introduced through the inlet ports, a mixing that is difficult to obtain in standard laminar flow conditions in devices used to date.

According to some embodiments, the transition between portions in each channel is via a channel segment that is perpendicular to the main plane. In other words, the portions of the channels that are defined in different planes are connected one to the other via channel segments that are oriented perpendicularly to the main plane.

According to other embodiments, the transition between portions in each channel is via a channel segment that is sloped with respect to the main plane.

By some embodiments, the device is made of a transparent material.

By some other embodiments, the device is made of a biocompatible polymer.

According to some embodiments, at least the inlet ports are configured to connect to fluid feed pumps. According to some other embodiments, the inlet ports and outlet ports are configured to connect to a fluid feeding and collecting system.

According to some embodiments, the biological sample holding chamber is configured to receive an auxiliary unit defining a plurality of sample wells, each well configured to hold a biological sample.

By some embodiments, the microfluidic device comprises at least one biological sample introduction port, linked to the biological sample holding chambers, for introducing the biological sample into the chambers. According to other embodiments, the microfluidic device can comprise a plurality of biological sample introduction ports, corresponding to the number of biological sample holding chambers, each biological sample introduction ports being in fluid communication with a corresponding biological sample holding chamber.

By some embodiments, the biological sample introduction port(s) is (are) configured to be linkable to a biological sample reservoir.

By some specific embodiments, the biological sample introduction port(s) is (are) configured to be linkable to a hanging drop unit. A hanging drop unit is a unit that permits growing of a biological sample (typically a cell cluster or spheroid) in a liquid that is pendent from an orifice or a concavity, such that the sample is grown in static conditions within the hanging drop. In the arrangement of this disclosure, according to some embodiments, the biological sample can be grown first in a hanging drop unit, and then transferred into the microfluidics device through biological sample introduction port(s) that are configured to interface with the hanging drop unit to safely introduce the biological sample to the microfluidic device.

According to some embodiments, the microfluidic devices of this disclosure are obtainable by the method disclosed herein.

According to other embodiments, the microfluidic devices of this disclosure are obtained by the method disclosed herein.

By some embodiments, the biological sample holding chambers are shaped as threaded cavities, and the device comprises threaded caps, configured to be threadingly received in said threaded cavities. Unlike standard microfluidic devices known to date, in which access to the sample holding chambers is limited-such arrangement permits easy access to the biological sample holding chambers, enabling closing and opening the chambers multiple times (or at will) by simply operating the threaded cap. The threading engagement of the threaded cavities and the threaded caps is configured to maintain a liquid-tight seal, such that no leakage occurs when fluids are fed (typically under pressure) into the device. Typically, the threaded caps are made of a transparent material, being the same or different from the material from which the device is made.

A device comprising such a cap is also an aspect of this disclosure. Hence, by another aspect, there is provided a microfluidic device suitable for maintaining a biological sample in viable conditions, the microfluidic device comprising:
at least one fluid inlet port and at least one fluid outlet port defining a fluid flow path therebetween;
at least one biological sample holding chamber shaped as a threaded cavity, and positioned between the at least one fluid inlet port and at least one fluid outlet port in said flow path;
at least one corresponding threaded cap, configured to be threadingly received in said threaded cavity;
an array of fluid feed channels, the channels linking between said at least one inlet port and said at least one biological sample holding chamber in said flow path; and
at least one fluid draining channel linking said at least one biological sample holding chamber to said at least one outlet port in said flow path.

According to some embodiments, the microfluidic device is made of a transparent material, typically of a polymeric material. According to other embodiments, the threaded cap is made of a transparent material, being the same or different from that of the microfluidic device.

According to some embodiments, the biological sample holding chamber is configured to receive an auxiliary unit defining a plurality of sample wells, each well configured to hold a biological sample.

By some embodiments, the threaded caps can be marked, colored or shaped to differentiate between them.

By some embodiments, the microfluidic device of this aspect is obtained by the method disclosed herein.

In another aspect, the present disclosure provides a kit comprising:
a microfluidic device suitable for maintaining a biological sample in viable conditions, the microfluidic device comprising at least one fluid inlet port and at least one fluid outlet port defining a fluid flow path therebetween; at least one biological sample holding chamber shaped as a threaded cavity, and positioned between the at least one fluid inlet port and at least one fluid outlet port in said flow path; an array of fluid feed channels, the channels linking between said at least one inlet port and said at least one biological sample holding chamber in said flow path; and at least one fluid draining channel linking said at least one biological sample holding chamber to said at least one outlet port in said flow path; and
at least one corresponding threaded cap, configured to be threadingly received in said threaded cavity.

By another aspect, there is provided a microfluidic system, comprising at least one microfluidic device as disclosed herein, and a plurality of fluid reservoirs each being in fluid communication with a corresponding inlet port of the microfluidic device.

The reservoirs can contain various fluids, e.g. solutions of different agents, diluent fluids, solutions of agents of different concentrations, etc.

By some embodiments, the system further comprises one or more pumps to pressure-feed said fluids from the reservoirs to the fluid inlet ports.

By some other embodiments, the system comprises one or more sensors for indicating one or more measurable parameters of the system or the biological sample.

By another aspect, this disclosure provides a method of determining a biological sample response to an environment ex vivo, the method comprising:
introducing the biological sample into a biological sample holding chamber of a microfluidic device as disclosed herein;
introducing one or more fluids into the flow path of the microfluidic device through the inlet port(s) to expose said biological sample to a desired environment; and
analyzing the response of said biological sample to said environment.

According to some embodiments, the microfluidic device comprises at least two ports, and the method comprises introducing a different fluid through each port.

By another aspect, this disclosure provides a method of screening a response of a biological sample ex vivo to at least one agent, the method comprising:
introducing the biological sample into a biological sample holding chamber of a microfluidic device as disclosed herein;
introducing one or more fluids containing said at least one agent into the flow path of the microfluidic device through the inlet port(s) to expose said biological sample to said at least one agent; and
analyzing the response of said biological sample to said agent.

According to some embodiments, the microfluidic device comprises at least two ports, and the method comprises introducing a different fluid through each port, each of the fluids containing a different agent.

According to some embodiments, the microfluidic device comprises a plurality of biological sample holding chamber, and the array of channels is configured to feed a different combination of agents or different concentration of said at least one agent to each of the chambers.

Such methods can be used, for example, for various scientific research purposes, medicinal drug screening assays and personalized medicine purposes.

As used herein, the singular form “a”, “an” and “the” include plural references unless the context clearly dictates otherwise. For example, the term “an inlet port” or “at least one inlet port” may independently include a plurality of inlet ports.

As used herein, the term “about” is meant to encompass deviation of ±10% from the specifically mentioned value of a parameter, such as temperature, concentration, length, diameter, etc.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any integer or step or group of integers and steps.

Generally it is noted that the term “ . . . at least one . . . ” as applied to any component of a composition of the invention should be read to encompass one, two, three, four, five, or more different occurrences of said component in devices or methods of this disclosure invention.

It is appreciated that certain features of this disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is an illustration of a 40-well mold printed onto a glass substrate according to a method of this disclosure. The wells' radius is 3.19 mm, and the height is 8 mm;

FIG. 1B shows 3 exemplary molds printed onto glass substrates, using different printing resins: Freeprint® (left), Miicraft™ BV007 (middle), and Luxaprint® (right), according to a method of this disclosure;

FIGS. 2A-2B show 4-[3-(4-Iodophenyl)-2-(4-nitrophenyl)-2H-5-tetrazolio]-1,3-benzene disulfonate (WST1) reagent: 24 h after seeding in the molds (FIG. 2A) and 72 h after seeding in the molds (FIG. 2B);

FIGS. 2C-2D is a comparison of viability of U87-MG, PC3M-LN4, BxPC3 and H460 cells after seeding in standard 96-well mold (“Control”) and Freeprint molds printed according to methods of this disclosure (“Mold”): 24 h incubation (FIG. 2C) and 96 h incubation (FIG. 2D);

FIGS. 3A-3C show a comparison between standard 2D polystyrene mold and 3D printed molds of this disclosure: PC3M-LN4 96 h after seeding (100,000 cells/well) in a standard 12-well plate (control) and in a 3D-printed Freeprint® well printed onto a 1 mm glass slide coated with PLL, scale bar=300 μm (FIG. 3A); PC3M-LN4 cells stained with Hoechst and Calcein AM and imaged 96 h after seeding, scale bar=500 μm (FIG. 3B); Images obtained 48 h after transfer of BxPC-3 spheroids to Freeprint® 3D-printed mold, standard polystyrene 12-well plate, scale bar=100 μm (FIG. 3C);

FIGS. 4A-4B show toxicity results and stained-imaging results, respectively, for H2286, PC3M-LN4, BxPC-3 and U87-MG cells seeded in printed Freeprint® (ResinF), BV-007A (ResinB) or Luxaprint® (ResinL) molds; scale bar=200 μm, n=6-8;

FIGS. 4C-4D show toxicity results for U87-MG, PC3M-LN4, BxPC-3 and H460 cells were seeded in standard regular 96-well plates (control) and 3D-printed molds containing the same diameter as the wells in the 96-well plates (radius=3.19 mm) for 24 h (FIG. 4C) and 96 h (FIG. 4D) incubation;

FIG. 4E shows viability of U87-MG, PC3M-LN4, BxPC-3 and H460 cells seeded in standard regular 12-well plates (control) and 3D-printed molds after 96 h incubation (100,000 cells/well);

FIGS. 4F-4G show U87-MG, PC3M-LN4, BxPC-3 and H460 cells seeded in 17.7 mm radius wells stained with Hoechst and Calcein AM, 96 h after seeding and imaged, scale bar=200 μm (FIG. 4F) and Calcein AM fluorescent intensity normalized to Hoechst fluorescent intensity (FIG. 4G), n=3-6;

FIG. 4H shows U87-MG, BxPC-3 and H460 seeded in 3D-printed molds or 96-well standard plates (control), spheroids stained after 96 h and 7 d with Calcein AM, scale bar=200 μm, n=3-4;

FIG. 4I shows SWT1 viability assay of U87-MG, BxPC-3 and H460 spheroids seeded in agarose, 96 h and 7 d after transfer to standard 96-well plates (control) and 3D-printed mold, n=3-4;

FIGS. 5A-5B are isometric views of a nut-shaped well according to an embodiment of this disclosure (FIG. 5A) suitable for fitting into a chip produced according to methods of this disclosure;

FIGS. 5C-5D are top views of the chip of FIG. 5B, without (FIG. 5C) and with the treaded cap well assembled into one of the sample-holding chambers (FIG. 5D);

FIGS. 6A-6C are views of a chip for obtaining a concertation gradient of fluids according to an embodiment of this disclosure: a top view (FIG. 6A), a transparent isometric view (FIG. 6B), and a close-up view of a fluid feed channel (FIG. 6C);

FIG. 6D is a picture of the chip of FIGS. 6A-6C, demonstrating the graduation of mixing of two fluids fed into the chip;

FIGS. 7A-7C are views of a chip for obtaining a concertation gradient of fluids according to another embodiment of this disclosure: a top view (FIG. 7A), a transparent isometric view (FIG. 7B), and a close-up view of a fluid feed channel (FIG. 7C);

FIG. 7D is a picture of the chip of FIGS. 7A-7C, demonstrating the graduation of mixing of two fluids fed into the chip;

FIG. 7E shows the absorbance measurements (2-570 nm) of blue-colored water and colorless distilled water fed into the chip at different relative concentrations, compared to standard pipetting dilution;

FIG. 8A shows UltraPure™ Agarose hydrogel microwells fabricated by casting into 3D-complementary templates printed according to methods of this disclosure—U87-MG, BxPC-3 and PC3M-LN4 4,000 cells/microwell were seeded immediately after staining with Cell Proliferation Staining Reagent Green Fluorescence Cytopainter and imaged after 24 h, scale bar=0.8 mm;

FIGS. 8B-8E show the construction of a “nut and bolt” chip according to an embodiment of this disclosure (FIG. 8B), with close-up views on the spheroid culture chamber in a “nut” shape (FIGS. 8C-8D) and the mixing and gradient concentration channels array (FIG. 8E);

FIG. 9A shows a 3D-printed mold containing 4 different hanging drop well geometries;

FIG. 9B shows bright-field and fluorescent images of U87-MG, PC3M-LN4, BxPC-3 cells stained with CellTrace™ CFSE and seeded 8,000 cells/60 μL drop—imaged 24 and 48 h after seeding, scale bar=300 μm;

FIGS. 9C-9D are, respectively, a top view of a chip prepared according to methods of this disclosure for receiving the hanging-drop unit of FIG. 9A, and an isometric view of the hanging drop unit and the chip before interfacing;

FIG. 10 shows a chip for maintaining a biological sample in hypoxic conditions prepared according to a method of this disclosure;

FIGS. 11A-11F are IC50 results for pancreatic cancer cells BxPC-3, PANC-1, and AsPC-1 to which 0, 1, 10, 50, 100 and 500 μM concentrations of chemotherapy treatments were added 24 h after seeding (IC50 values normalized so that the highest inhibition induced is considered as 100%);

FIGS. 12A-12I show viability test results for various chemotherapy treatments for spheroids obtained from patients: Pancreatic adenocarcinoma (T1, FIG. 12A), Desmoplastic small round cell tumor of the peritoneum (T2, FIG. 12B), Primary peritoneal carcinoma (T3, FIG. 12C), Moderately differentiated adenocarcinoma of large intestine (T4, FIG. 12D), Mucinous carcinoma of appendix (T6, FIG. 12E), Squamous cell carcinoma of the anal canal (T7, FIG. 12F), Pancreatic adenocarcinoma (T8, FIG. 12G), Pancreatic neuroendocrine carcinoma (T9, FIG. 12H), and Adenocarcinoma of colon (T10, FIG. 12H);

FIGS. 13A-13B show viability assay performed on T5 patient-derived spheroids 7 dafter treatment initiation (FIG. 13A), and spheroids formed from cells extracted from a tumor sample obtained from patient T5 (FIG. 13B);

FIGS. 14A-14B show analysis of spheroid area 48 h after seeding (depicted as 0 h) relative to spheroid area 24 h after the addition of treatment (FIG. 14A), and WST1 viability assay was performed on BeWo spheroids 24 h after the addition of treatments (FIG. 14B);

FIG. 15A-15B show cisplatin treatment effect on viability (FIG. 15A) and spheroids area expansion on day 5 relative to 72 h after seeding (FIG. 15B).

### DETAILED DESCRIPTION OF EMBODIMENTS

Given the poor successful rates of experimental drugs in clinical trials, fully humanized ex vivo models are becoming the preferred approach for disease modelling and drug development. Despite the clear advantages of organ on chip technology, this field endures major drawbacks that limit its broader implantation, mostly due to chip materials and the long and laborious fabrication processes.

The examples below demonstrate methods and devices of this disclosure, that provide a cost-effective solution for obtaining fully 3D-printed devices that are transparent, biocompatible, versatile, and sample accessible. The devices (or chips) of this disclosure are useful, for example, in personalized medicine, allowing more precise prediction via drug efficacy in tissue-like physiological conditions.

All 3D-printed molds and microfluidic chips described below were fabricated using a digital light processing stereolithography printer Asiga Max (Sydney, Australia) with a LED light source of 385 nm UV wavelength. The molds and microfluidic chips intended for cell culture were all printed from Freeprint®-ortho unless specifically otherwise mentioned.

Statistical data was analyzed on GraphPad Prism 9 (www.graphpad.com, San Diego CA) and all experiments had at least three independent replicates. Studies containing two groups were assessed using the unpaired two-tailed Student's t-test. Studies containing more than three groups were compared and analyzed using a one-way analysis of variance (ANOVA), and significant differences were detected using Tuckey's multiple comparison post-test. Differences were considered statistically significant for p<0.05.

### Example 1: Preparation of Chip by Direct Printing of Resin on Glass

A glass slide was used as a substrate for printing to provide maximal optical transparency. The glass slide was dipped in 2% TMSPMA (3-(trimethoxysilyl) propyl methacrylate) in ethanol for 2 minutes in darkness conditions to permit absorption of the TMSPMA onto the surface of the glass. The slide was then immersed in pure ethanol for 2 minutes in dark conditions, and then placed in an oven (105° C.) to fixate the TMSPMA onto the glass to form a hydrophilic coating.

The coated glass substrate was placed in the printer, and a first layer of a UV-curable clear polymer was then printed onto the substrate, typically in a thickness of about 0.01 mm and then cured by operating UV-LED light for 5 minutes. The ensuing resin layers, having a thickness of at least an order of magnitude larger than the first layer (i.e. at least 0.1 mm) were then printed on top, and at least partially cured layer-by-layer, according to the designed geometry of the device is obtained.

The device was then washed one or more times with pure ethanol (or isopropanol) and left to dry and then cured using UV light for 60 minutes. After curing, the device was emersed in ethanol for at least 12 hours in order to leach out unreacted monomers and/or curing initiators/catalysts.

### Example 2: Selection of Resin for Cell Culturing

Most resins used as inks in DLP are not transparent enough for receiving high resolution microscopy images. This is partly due to the material properties, but also due to the voxel resolution of the printing process.

To overcome this, the printing method described in Example 1 was developed, that permits direct printing onto hydrophilic surfaces, such activated glass.

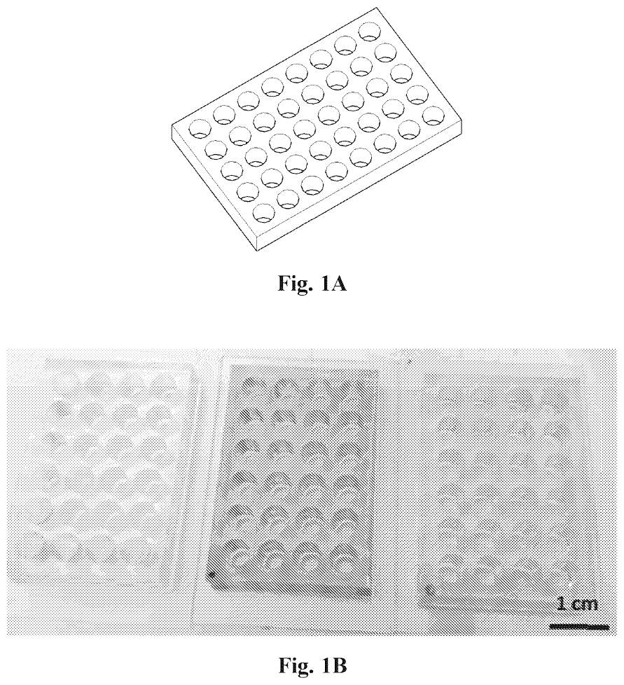

Three clear resins BV007 (Miicraft, Jena, Germany) Luxaprint® (Detax GmbH) and Freeprint® (Detax GmbH) were used to print 96-well plates as a model mold. The resins were printed according to the method described in Example 1. The molds were sterilized in a biological hood using UV for 30 min. The design of the mold and the resulting printed molds can be seen in FIGS. 1A and 1B, respectively.

Human glioblastoma U87-MG, prostate PC3M-LN4, lung H460 and H2286, placenta BeWo, and pancreatic cancer cell lines BxPC-3, PANC-1, and AsPC-1 were obtained from ATCC (VA, USA). All cells were maintained in a 10% fetal calf serum (FCS) medium with Penicillin/Streptomycin (P/S) and kept in a humidified incubator at 37° C. with 5% CO₂ unless otherwise specified. For U87-MG cell lines, EMEM (Biological Industries, Israel) supplemented with 1% sodium pyruvate (Life Technologies, MA, USA) and 1% Glutamine (Life Technologies) was used. PC3M-LN4, H460, H2286, BxPC-3 and AsPC-1 cells were maintained in RPMI-1640 (Life Technologies). PANC-1 cells were grown in DMEM (Life Technologies). BeWo cells were grown in F-12K medium (ATCC) supplemented with 0.1% FCS. Patient-derived cells were isolated from cancer tissues by sectioning the tissue into ˜1 mm pieces and digesting for 60-90 minutes at 37° C. with 5% CO₂, while slowly stirring with a magnet in DMEM/F12 medium (Life Technologies) supplemented with P/S and 0.14 Wunsch units/ml of Liberase™ Research Grade (Roche Diagnostics). Digested tissue was filtered through a cell strainer, after which supernatant was diluted with a stop reaction medium: DMEM/F12 supplemented with P/S and 20% FCS and centrifuged at 1,200 RPM for 5 minutes. Cells were plated and maintained in RPMI:F12:DMEM medium (1:1:3) supplemented with 10% FCS, 1% glutamine, P/S, HEPES (Biological Industries), 1% hydrocortisone (Sigma-Aldrich, MO, USA) and epithelial growth factor (EGF) 5 ng/ml (ProSpec, Israel).

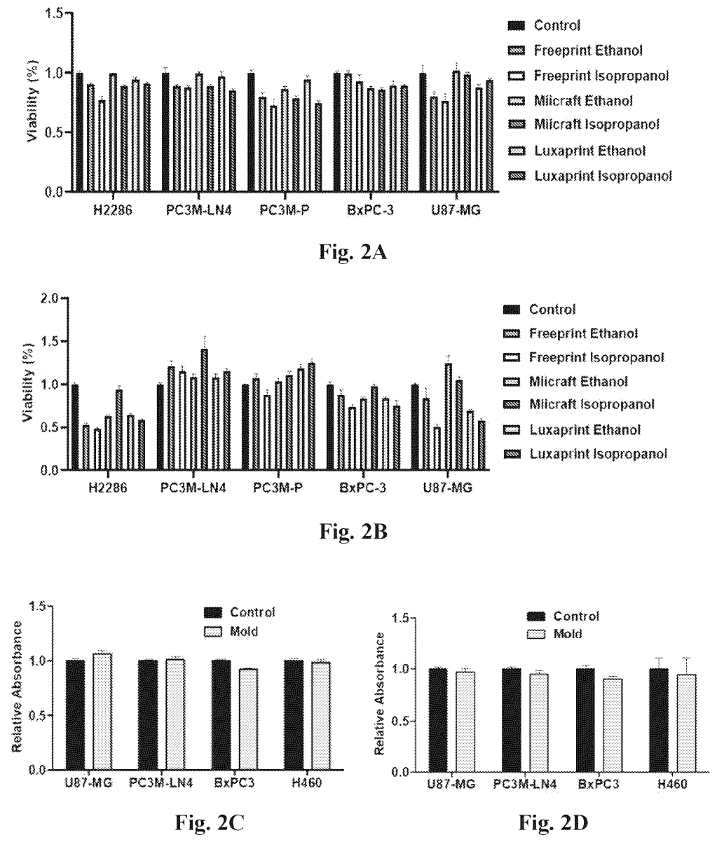

Prior to cell seeding, the molds were coated with poly-L-lysin (PLL) for 1 h, and then rinsed 3 times with phosphate-buffered saline (PBS). H2286, PC3M-LN4, PC3M-P, BxPC-3 and U87-MG cells were seeded in the wells. WST1 (4-[3-(4-Iodophenyl)-2-(4-nitrophenyl)-2H-5-tetrazolio]-1,3-benzene disulfonate) was added after 24 h and 72 h in order to test cell viability. The absorbance was measured at 480 nm using a plate reader, as can be seen in FIGS. 2A and 2B, respectively. All resins showed sufficient viability of the cells.

Toxicity assay was carried out for the Freeprint® molds. The molds were immersed in ethanol for 20 min and then sonicated in ethanol at 20% for 5 min, and then placed in UV for 1 h and immersed in ethanol again overnight. The molds were removed from the ethanol and placed in distilled water for 30 min and then placed in the biological hood under UV for 30 min for sterilization. Next, the sterilized molds were coated with PLL (50 μL/well) for 1 h at room temperature. The molds were rinsed with PBS 4 times (on the 4^{th} time the molds were placed in the incubator for 1 h). U87-MG, PC3M-LN4, BxPC3 and H460 cells were seeded in the molds and in regular 96-well plates (as control) for 24 h and 96 h incubation (10,000 cells/well and 3,000 cells/well, respectively, 100 μL per well). After 24 h and 96 h, WST1 was added 1:10 for 45 min. into each well and absorbance was read at 450 nm. The results are provided in FIGS. 2C-2D. As can be seen, no statistical difference was observed between the 3D-printed molds and control standard 96-wells, thereby shoring the biocompatibility of the molds when prepared according to a method of this disclosure.

### Example 3: Comparison Between 2D and 3D Mold

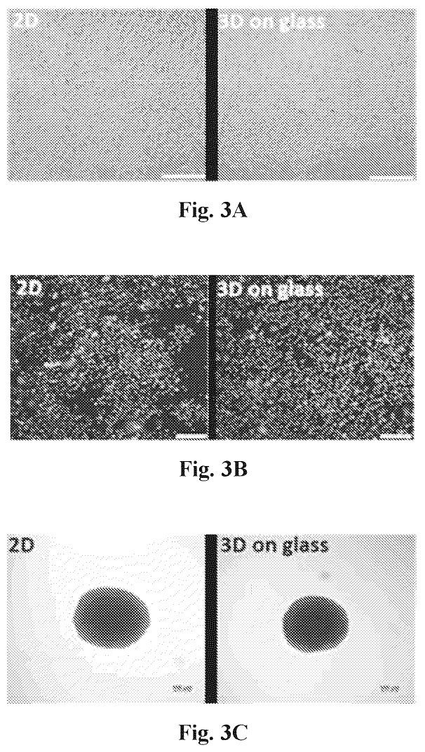

Images of PC3M-LN4 cells and BxPC-3 spheroids seeded in standard polystyrene dish plates and 3D-printed molds on glass are presented in FIGS. 3A-3C. The optical transparency of cells and spheroids seeded in devices printed onto glass slides is much higher, as imaged by brightfield and fluorescent microscopes.

### Example 4: Biocompatibility of 3D-Printed Molds in Long-Term Culture of Cells and Multicellular Spheroids

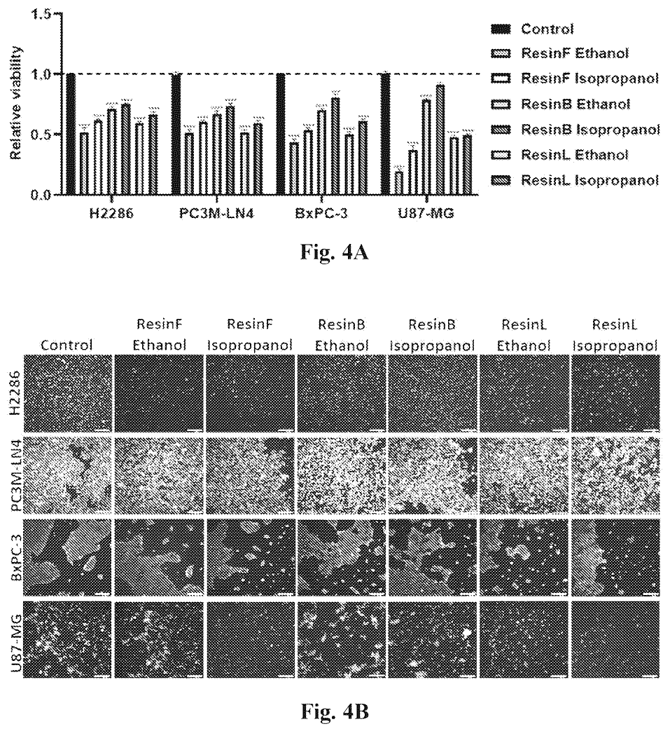

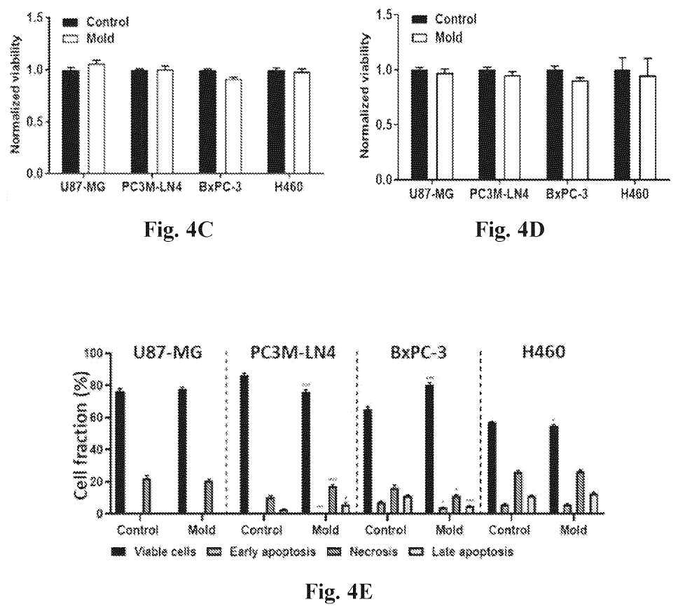

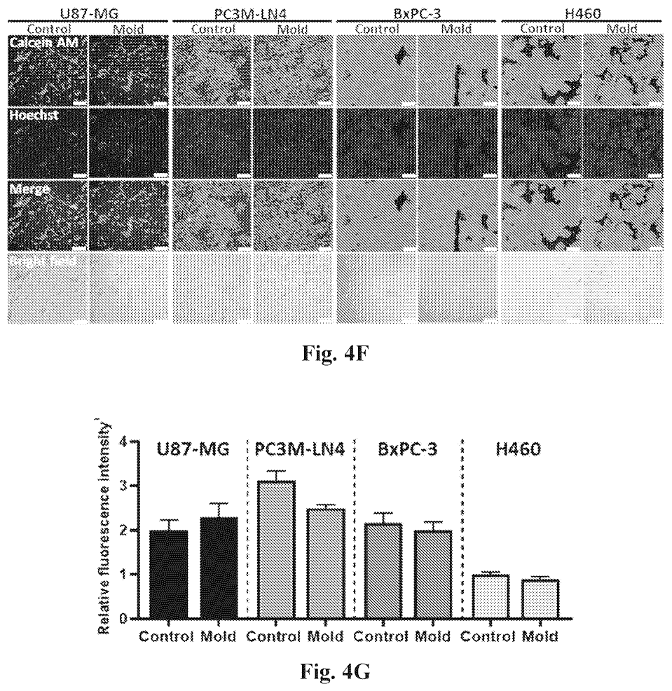

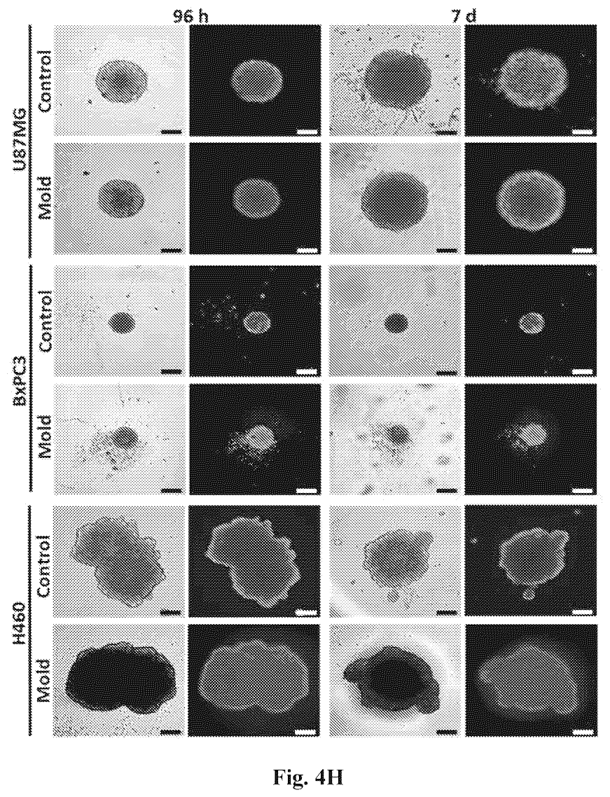

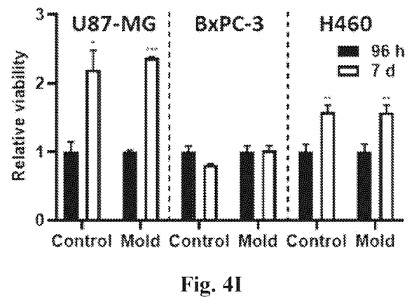

As photocurable resins are widely considered to be toxic to cells the biocompatibility of the 3D-printed resins prepared by a method of this disclosure was investigated for cell and spheroid culturing. FIGS. 4A-4B show the cytotoxicity of the resins used for printing molds for cell culture.

FIGS. 4C-4D show that there is no significant difference in the viability and proliferation of U87-MG, PC3M-LN4, BxPC-3 and H460 cells, 24 and 96 h after their seeding in standard 96-well plates or 3D-printed molds on glass slides with the same dimensions, when using our protocol. Flow cytometry analyses of apoptosis and necrosis using Annexin-V-APC and 7-AAD staining of U87-MG, PC3M-LN4, BxPC-3 and H460 cells 96 h after seeding revealed no significant difference in the cell fraction of U87-MG cells in the different cell cycle stages, while PC3M-LN4 cells showed no more than 10% reduction in the fraction of viable cells seeded in 3D-printed molds compared with control standard wells (FIG. 4E). In contrast, BxPC-3 cells cultured in 3D-printed molds showed an increase of 15% in the fraction of viable cells compared with standard control wells. H460 cells displayed a less than 3% difference in the fraction of viable cells between the two conditions.

The flow cytometry results were further supported by cell staining with Calcein AM and Hoechst. Calcein AM fluorescent intensity was normalized to the fluorescent intensity of Hoechst nuclei staining and revealed no significant difference in the viability (FIGS. 4F-4G). Further validation for maintaining proper viability of cells was done by monitoring U87-MG, BxPC-3 and H460 spheroids with Calcein AM 96 h and 7 d after their transfer to either a 96-well standard plate or its equivalent 3D-printed mold. As seen in FIG. 4H, similar trends in spheroid viability in both conditions was observed over time. Spheroids' viability was also quantified 7 d after their transfer using WST1 reagent (FIG. 4I). U87-MG and H460 spheroids displayed similar increases in viability over time in both conditions, BxPC-3 spheroids remained with the same levels of viability over time in both conditions.

### Example 5: Chips with Shaped Wells Having Treaded Capping

The ability to access tissue samples after they are inserted into microfluidic chips is crucial for in-depth analysis and for dynamic analysis over time. Many works have attempted to tackle this matter using two main approaches: extracting the biological sample or disassembling the microfluidic device. Many microfluidic devices are fabricated in such a way that the different layers comprising the devices are sealed together forming a “black box”, making it difficult to reach and analyze the cultured samples. In other words, in order to reach the samples there is need to either extract the biological sample, or the device itself needs to be dismantled.

Hence, a device (or chip) configuration fabricated in a method of this disclosure, i.e. the “nut-and-bolt” configuration, was designed to permit easy access to the biological sample and multiple opening and/or closing of the chip, without disrupting the course of the experiment. In contrast to many works that utilized devices containing bolts or clamps to fasten the systems, in the presently exemplified design the culturing wells themselves function as a “nut and bolt”, granting access to the biological samples for extraction and return at any given moment without disrupting the course of the experiment. Particularly, apart from the reusability of this platform for multiple experiments, the design allows access to the sample multiple times for long-term tracking, guarantying optimal sealing even while the experiment is still running. Furthermore, since the biological sample size is different for each type of sample taken from a patient, the methods described herein permit endless versatility in size and shape.

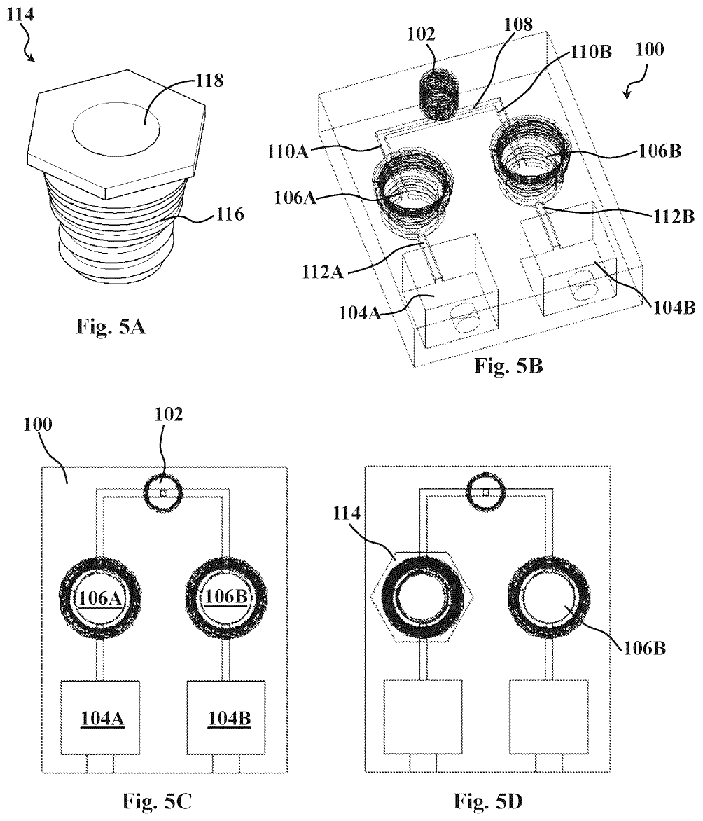

In the “nut-and-bolt” configuration, seen in FIGS. 5A-5D, chip 100 contains a fluid inlet port 102 and two fluid outlet ports 104A,104B defining a fluid flow path therebetween. In this example, two biological sample holding chambers, 106A,106B, are positioned between the fluid inlet port and the fluid outlet port in the flow path. An array of fluid feed channels links between the inlet port and the biological sample holding chambers, and includes a distribution manifold, and a set of first fluid feed channels 110A,110B, each corresponding to the biological sample holding chambers, 106A,106B and configured to feed the fluid thereto. Two fluid draining channels 112A,112B link each of the biological sample holding chambers to its respective outlet port 104A,104B.

Each of the biological sample holding chambers, 106A,106B is shaped as a threaded cavity, that is configured to receive a corresponding threaded cap 114, shown in detail in FIG. 5A. Cap 114 has threading 116 that matches that of the threading in the biological sample holding chambers. Hence, the chambers can be closed by threading the cap thereinto (as seen in FIG. 5D, in which chamber 106A is closed by the cap 114), and opened by threading the cap out of the cavity. This permits easy access at will to the biological sample placed within the chamber. The cap 114 can have a transparent window 118, or can be made entirely from a transparent material, thereby permitting visual inspection or optical analysis of the sample within the chamber.

The cap may be color-coded, marked or have various shapes (in this specific example the top of the cap has a hexagonal shape, however the cap can have any other shape), or any other suitable marking, in order to differentiate between different examples once the caps are threaded into the cavities of the chambers.

### Example 6: Chips with Graduated Channels

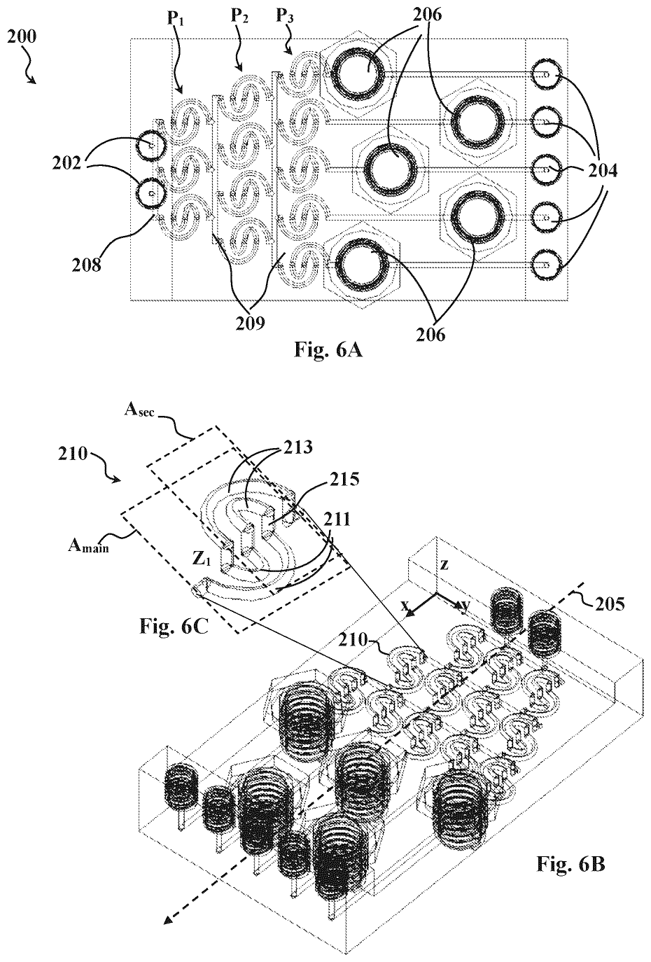

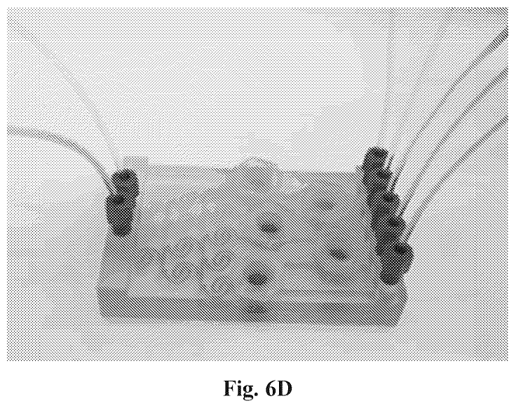

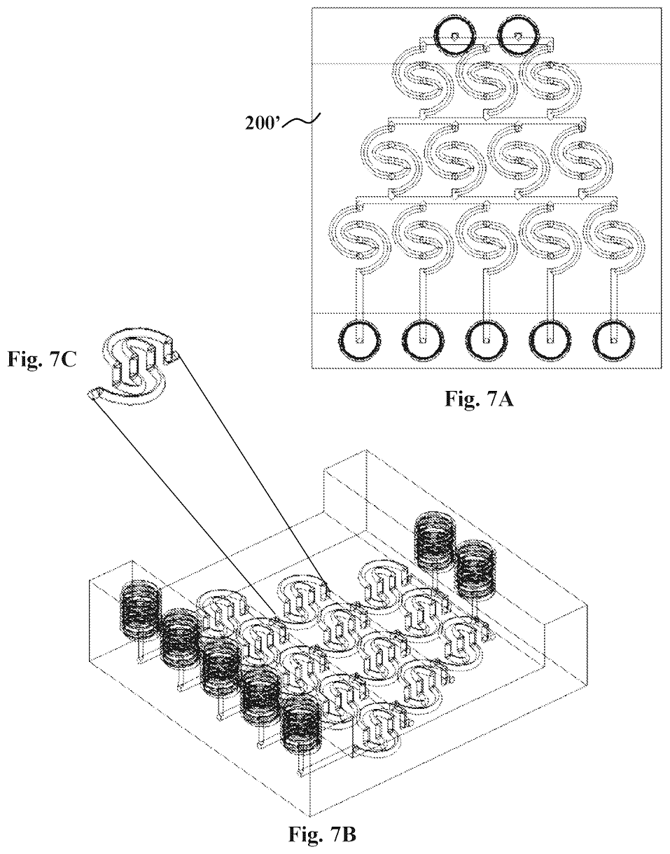

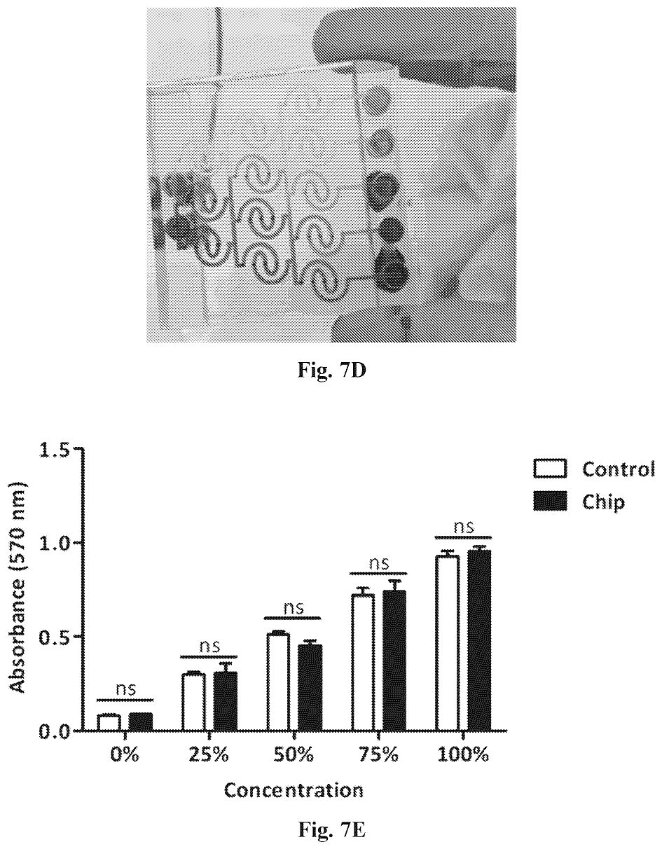

FIGS. 6A-6D and 7A-7E exemplify devices that can be utilized to form a gradient of concentrations of an agent, thereby exposing the biological sample to gradually decreasing concentration (or relative concentration) of one or more agents or one or more fluids.

In the specific example of FIGS. 6A-7E, device 200 includes two fluid inlets 202 (i.e. n=2), for example one for feeding liquid that comprises a given concentration of a therapeutic agent, and the other for feeding a diluting liquid, with the aim of eventually feeding different concentrations of the therapeutic agent to each of the biological sample holding chambers 206.

The device 200 includes three sets of fluid feed channels (P₁,P₂,P₃), namely i=3, and hence five fluid outlet ports (n+i) 204, and five biological sample containing chambers 206. The inlet ports and the outlet ports define between them the flow path, and also define a general axial direction 205 of the device.

The first set of channels P₁ contains n+1 (i=1) channels, the second set of channels P₂ contains n+2 channels (i=2) and the third set of channels P₃ contains n+3 channels (i=3). Therefore, in the present example, P₁ contains 3 channels, P₂ contains 4 channels, and P₃ contains 5 channels.

Inlet ports 202 are connected to the first set of channels P₁ by distribution manifold 208, and two adjacent sets are connected to each other by a collection channel 209. The number of collection channels is i−1, namely in this example the number of collection channels is two (given that i=3).

The sets of channels are preferably arranged in a staggered manner along the main plane Aₘₐᵢₙ. In other words, the channels of P₁ are arranged off-set from the channels of P₂, and the channels of P₂ are arranged off-set from the channels of P₃. This allows for formation of concentration gradient of or mixing ratios between fluids, each collecting channel receiving fluids from a previous channels set and distributing the fluids to the ensuing channels set, thereby forcing fluids introduced from a pervious set to be mixed in the ensuing set of channels. For example, in the exemplified device, P₁ contains 3 channels: a first channel is be fed 100% of the first fluid through the manifold, a second channel will be fed a 50%-50% of the first and second fluids, while the third channel will be fed 100% of the second fluid. The fluids from P₁ are further mixed therebetween in P₂, and from there further mixed in P₃—eventually resulting in 5 different relative concentrations of the two fluids that are each fed to a corresponding biological sample holding chamber 206: 100% first fluid, 25%±75%, 50%±50%, 75%±25% and 100% of the second fluid. In this manner, the structure of the device permits exposure of the biological samples to a variety of different environments in a compact and highly tailorable arrangement.

As better seen in FIGS. 6B-6C, each fluid feed channel 210 in the sets P₁, P₂ and P₃, has one or more portions 211 thereof defined in main plane Aₘₐᵢₙ, and one or more portions 213 defined along plane Aₛₑₑ that is vertically distanced (i.e. along the z-direction) from the main plane. Connecting between portions 211 and 213 are segments 215 which are directed generally perpendicularly to the main plane Aₘₐᵢₙ. Segments 215 have a length Z₁, that corresponds to the distance between Aₘₐᵢₙ and Aₛₑₑ.

Thus, by the arrangement of FIGS. 6A-6C, the two fluids introduced into the device are intermixed at different ratios in the sets of channels, thereby permitting introduction of a different composition of fluids into each of the sample holding chambers 206.

FIGS. 7A-7E demonstrate another version of device 200, namely device 200′, with the sample holding chambers 206 absent. This device permits drawing of the resultant different composition fluids from the outlet ports for use in other devices-namely the device shown in FIGS. 7A-7E can be utilized in order to prepare various mixtures of fluids for further use.

While the present examples are based on devices in which n=2 and i=1 to 3, it is understood that any other number of n and i can be utilized.

### Example 7: 3D-Printed “Nut and Bolt” Chip for Spheroid Culture Using the Ultra-Low Attachment Surface Method

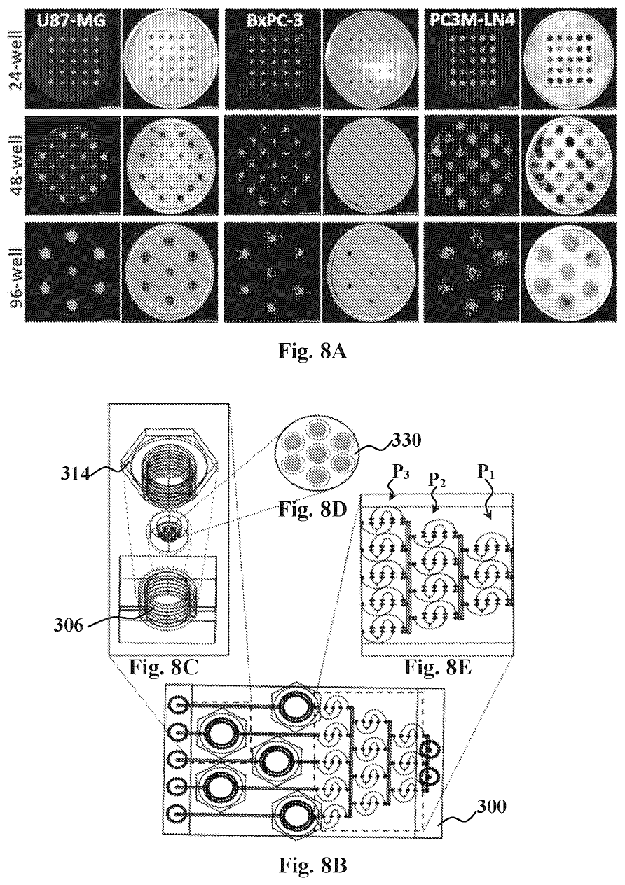

A common method to create spheroids is using ultra-low attachment hydrogel surfaces. For this approach of spheroid assembly, templates containing the complementary geometry of the microwells were designed in 3 different variations using methods of this disclosure. The agarose hydrogel arrays were fabricated as the complementary of the printed molds and placed in 96, 48 and 24-well standard plates, respectively. U87-MG, BxPC-3 and PC3M-LN4 cells were stained with Cell Proliferation Staining Reagent Green Fluorescence Cytopainter and seeded 4,000 cells/microwell. The wells were imaged 24 h after cell seeding, showing the formation of homogenous viable spheroids (FIG. 8A). In order to obtain a concentration gradient flow on a chip 300, spiral channels sets P₁, P₂ and P₃ extended over several planes were designed to maximize mixing in a minimal XY axis area (FIGS. 8B-8E). The chip design (FIG. 8B) shows the sample chambers 306 intended for spheroid culture in the shape of threaded cavities (a “nut”), allowing repeated opening and closing of the chamber and convenient access to the biological samples when removing the threaded cap 314 (a “bolt”, FIG. 8C), while obtaining complete sealing and a leakproof device when screwing it back shut. The cavity is designed to hold a wells' unit 330 containing several microwells (FIG. 8D) that was placed in each sample culture chamber, thus enabling the growth of numerous spheroids under various conditions. The channels array (FIG. 8E) permits generation of a concentration gradient that the culture chambers are exposed to under a laminar flow profile. It was observed that the flow velocity rises and falls alternately in the spiral channels extending over several plains, thus accomplishing effective mixing.

### Example 8: Spheroid Culturing in Microfluidic Device Using the Hanging Drops Technique

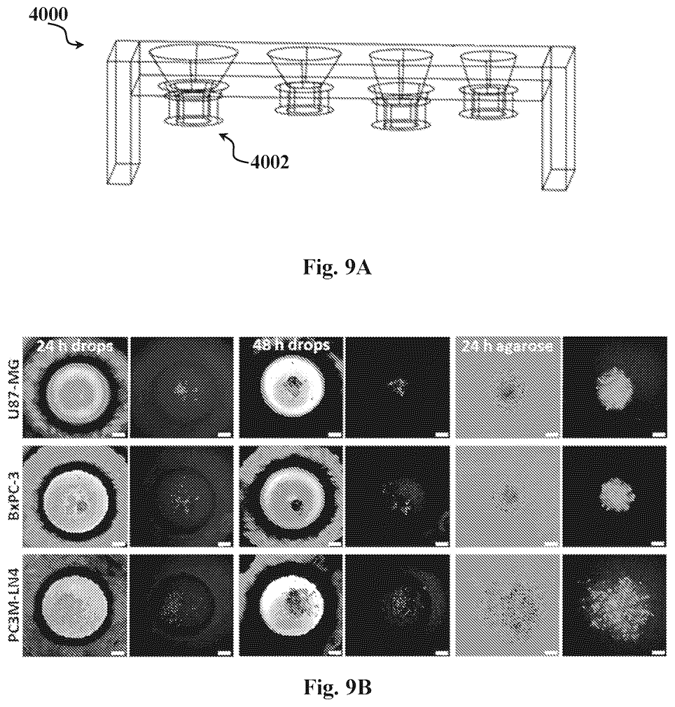

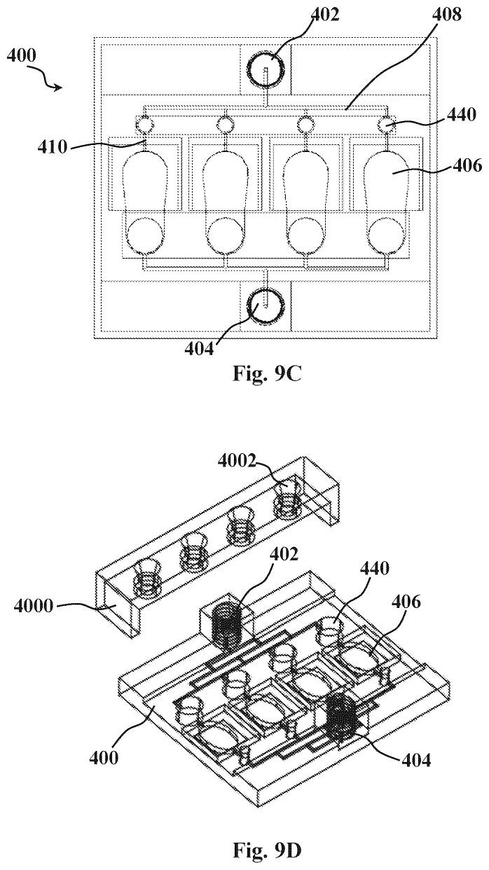

One of the main methods used to create spheroids is the hanging drop technique. Using the methods of this disclosure, hanging drop unit 4000 containing hollow wells 4002 of different geometries was 3D-printed, as seen in FIG. 9A. The well geometry was designed to maintain the most stable drops under shaking. In unit, the drops held for 5 days without the need to replenish the media when placed in a humidified 37° C. incubator. In order to prove that the geometry of the device supports the formation of viable spheroids in stable drops, U87-MG, PC3M-LN4, BxPC-3 cells stained with CellTrace™ CFSE were seeded and monitored. Viable spheroids were formed 3 days after their initial seeding as indicated by the green-fluorescent images (FIG. 9B).

A microfluidic chip 400 was designed and prepared according to methods of this disclosure, to enable the convenient transfer of the drops containing the spheroids into a biological sample introduction port 440 defined in device 400. Each drop is transferred individually into an introduction port 440 for the purpose of long-term culturing of the spheroids under flow conditions (FIG. 9C-9D), from which it is introduced into the sample holding chambers 406 by the flow of the fluid from inlet port 402 towards outlet port 404. The chip was designed to form laminar flow profiles within the spheroid-holding chambers to ensure proper shear forces development within the chambers as to minimize shear force effects on the spheroids.

### Example 9: Hypoxic Chip

Performing experiments under hypoxic conditions is crucial for several scientific disciplines. Hypoxia, or low oxygen levels, is associated with several physiological and pathological conditions in humans. By simulating hypoxic conditions in controlled laboratory settings, researchers can investigate the adaptive responses and mechanisms involved in cellular and organismal responses to oxygen deprivation. This helps unravel the underlying molecular pathways involved in diseases such as cancer, cardiovascular disorders, and neurological conditions. Furthermore, studying hypoxia can contribute to the development of novel therapeutic strategies aimed at mitigating the harmful effects of oxygen deprivation. The utilization of 3D-printed chips for creating hypoxic conditions offers several advantages over traditional PDMS (Polydimethylsiloxane) chips. 3D-printed chips offer better gas permeability control, enabling accurate regulation of oxygen levels within the chip's microenvironment. This ensures the creation of stable and reproducible hypoxic conditions, crucial for establishing hypoxic conditions in research, with broad applications in various fields including cell biology, tissue engineering, and drug discovery. The hypoxic chip can be sealed to maintain the desired oxygen level, and a controlled fluid containing a specific amount of oxygen can be introduced into the chamber. This ensures that the oxygen level remains regulated and consistent within the chamber throughout the experiment.

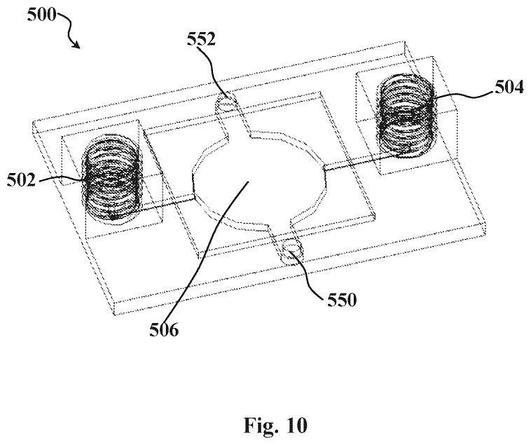

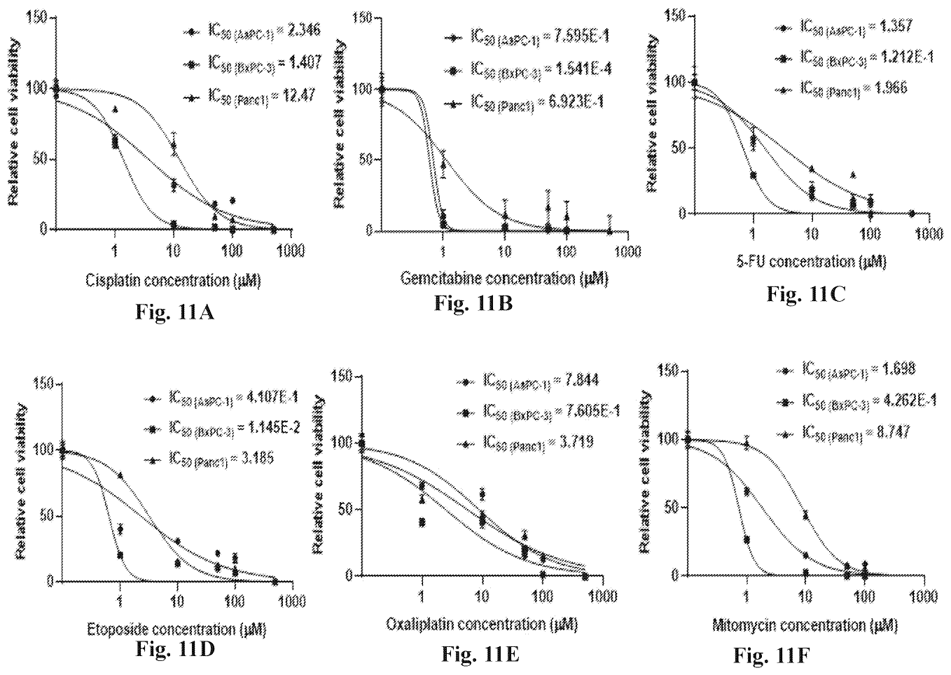

An example for such a hypoxic microfluidic chip is shown in FIG. 10, which can be printed by methods of this disclosure. Chip 500 comprises inlet and outlet ports 502 and 504, respectively, with a sample holding chamber 506 defined in a flow path therebetween. Further defined are auxiliary ports 550 and 552, through which the oxygen level in the chip can be controlled.

### Example 10: Chemotherapy Responses of Patient-Derived Multicellular Tumor Spheroids

The diversity and plasticity within tumors, combined with patient-specific factors, results in different therapeutic outcomes for the same treatments, leading to the need of tailored specific treatments. For this purpose, the development of reliable 3D tumor models comprised of the patient's own cells is of great value.

To confirm the use of the chip as an ex-vivo hosting device for personalized therapy, various tumor samples were collected from ten patients: Pancreatic adenocarcinoma (T1, T5, T8), Desmoplastic small round cell tumor of the peritoneum (T2), Primary peritoneal carcinoma (T3), Moderately differentiated adenocarcinoma of large intestine (T4), Mucinous carcinoma of appendix (T6), Squamous cell carcinoma of the anal canal (T7), Pancreatic neuroendocrine carcinoma (T9) and Adenocarcinoma of colon (T10). Table 1 details patients' profiles and their responses to therapies.

Each fresh tissue was processed, and patient-derived 3D multicellular tumor spheroids were formed and treated with different chemotherapy combinations according to the following protocol: cells were seeded 8,000 cells/well in 96-well wells in chips prepared according to a method of this disclosure. 24 h after seeding, chemotherapy treatments were added using 0, 1, 10, 50, 100 and 500 μM concentrations and left for 72 h followed by the WST1 assay conducted as previously mentioned. The chemotherapy used were Oxaliplatin, Gemcitabine, Etoposide, Mitomycin, 5-FU, Cisplatin and Bevacizumab. The last provided a negative control as its anticancer activity was accomplished mainly via inhibition of angiogenesis. The spheroids were imaged again 7 d after adding the treatments and their area was measured and analyzed. WST1 reagent was used to measure viability.

TABLE 1
Patients' profiles and responses to therapies

| | | Age | | | Treatment and response (<2 years) | |
|---|---|---|---|---|---|---|
| | | Gender | Sample | Genomic | Prior to | |
| | Diagnosis | Stage | site | finding | dissection | Post dissection |
| T1 | Pancreatic | 55 | Secondary- | BRCA1, KRAS, | — | 1. Short term partial |
| | adenocarcinoma | Male | peritoneum | MTAP, | | remission with Folinic acid, |
| | | IV | | CDKN2A/B | | 5-FU, irinotecan, oxaliplatin |
| | | | | | | 2. Stable disease with |
| | | | | | | pembrolizumab and |
| | | | | | | lenvatinib |
| T2 | Desmoplastic | 27 | Secondary- | No alterations | — | 1. Stable disease with |
| | small round cell | Male | peritoneum | detected | | vincristine, doxorubicin, |
| | tumor of the | IV | | | | cyclophosphamide, |
| | peritoneum | | | | | ifosfamide and etoposide |
| | | | | | | 2. Stable disease with |
| | | | | | | irinotecan and temozolomide |
| | | | | | | 3. Deterioration with |
| | | | | | | pembrolizumab |
| | | | | | | 4. Stable disease with |
| | | | | | | cyclophosphamide and |
| | | | | | | topotecan |
| T3 | Primary | 67 | Primary | TP53, PIK3CA, | 1. Partial | Deterioration with |
| | peritoneal | Female | | MET, BRCA2 | remission with | topotecan; deceased |
| | carcinoma | IV | | | paclitaxel and | |
| | | | | | carboplatin | |
| | | | | | 2. Short term | |
| | | | | | stable disease | |
| | | | | | with bevacizumab | |
| | | | | | and doxil | |
| | | | | | 3. No response | |
| | | | | | with Olaparib | |
| T4 | Moderately | 70 | Secondary- | TP53, KRAS, | Partial response | Deceased (not cancer |
| | differentiated | Male | liver | PIK3K, SMAD4 | with folinic acid, | related) |
| | adenocarcinoma | IV | | | 5-FU and | |
| | of large intestine | | | | oxaliplatic | |
| T5 | Pancreatic | 71 Male | Primary | TP53, KRAS, | — | Deceased |
| | adenocarcinoma | III | | PIK3K, SMAD4 | | |
| T6 | Mucinous | 65 | Secondary- | ATM | — | 1. Stable disease with |
| | carcinoma of | Female | peritoneum | | | oxaliplatin and 5-FU |
| | appendix | IV | | | | 2. Stable disease with |
| | | | | | | bevacizumab and 5-FU |
| T7 | Squamous cell | 70 | Secondary- | KRAS, SMAD4, | 1. Partial | 1. Partial remission with |
| | carcinoma of the | Male | liver | PIK3CA, MYC, | remission with | paclitaxel |
| | anal canal | IV | | BRCA1, EZH2, | cisplatin and 5-FU | 2. Deterioration with |
| | | | | FABP5, PARP1, | 2. Stable disease | mitomycin |
| | | | | TOPOila, BIRC5 | with nivolumab | |
| | | | | | 3. Partial | |
| | | | | | remission with | |
| | | | | | paclitaxel | |
| T8 | Pancreatic | 44 | Primary | TP53, BRCA2, | Stable disease | Partial response with |
| | adenocarcinoma | Male | | KRAS, | with folinic acid, | gemcitabine and cisplatin |
| | | III | | CDKN2A/B | 5-FU, irinotecan | |
| | | | | | and oxaliplatin | |
| T9 | Pancreatic | 56 | Primary | — | — | Somatostatin, cancer free |
| | neuroendocrine | Male | | | | |
| | carcinoma | III | | | | |
| T10 | Adenocarcinoma | 53 | Secondary- | TP53, HER2+, | — | 1. Remission with oxaliplatin, |
| | of the colon | Female | peritoneum | APC | | capecitabine and Herceptin |
| | | IV | | | | 2. Remission with herceptin |

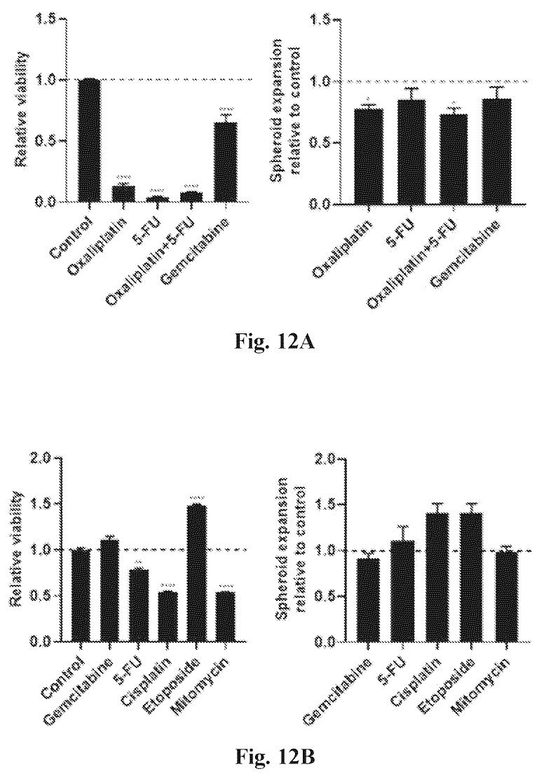

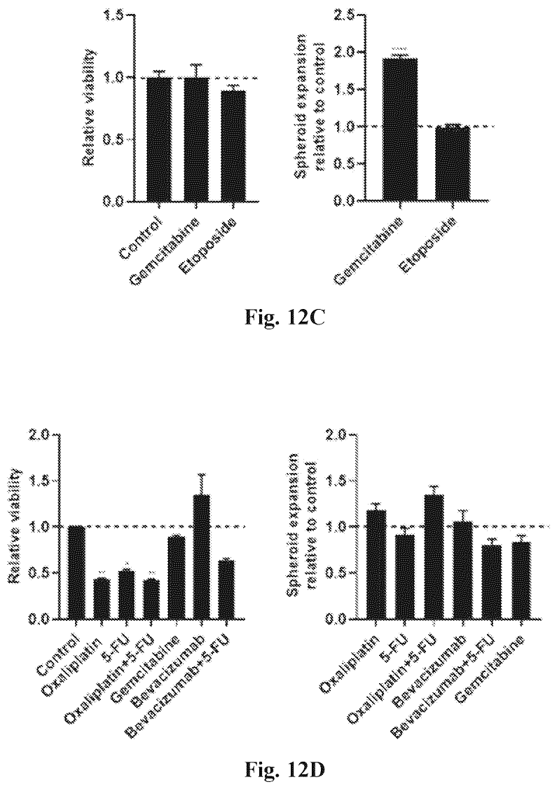

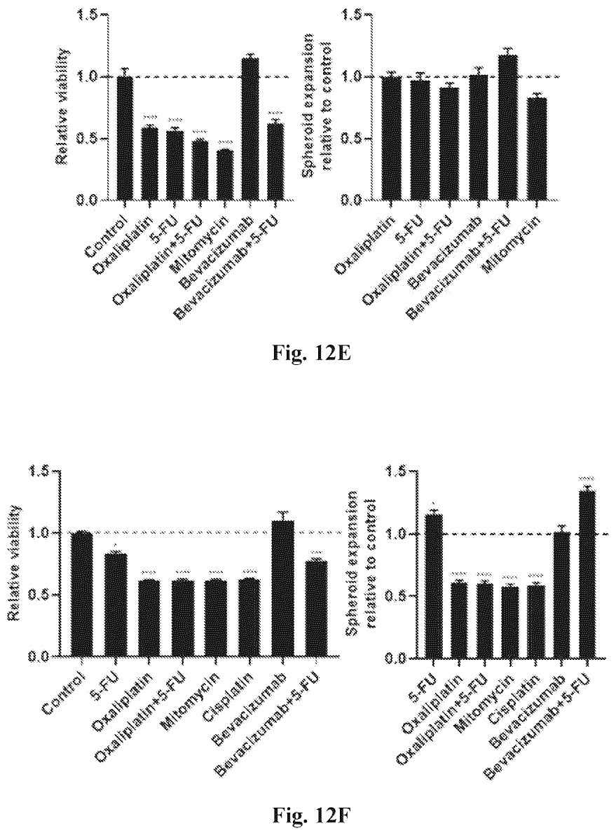

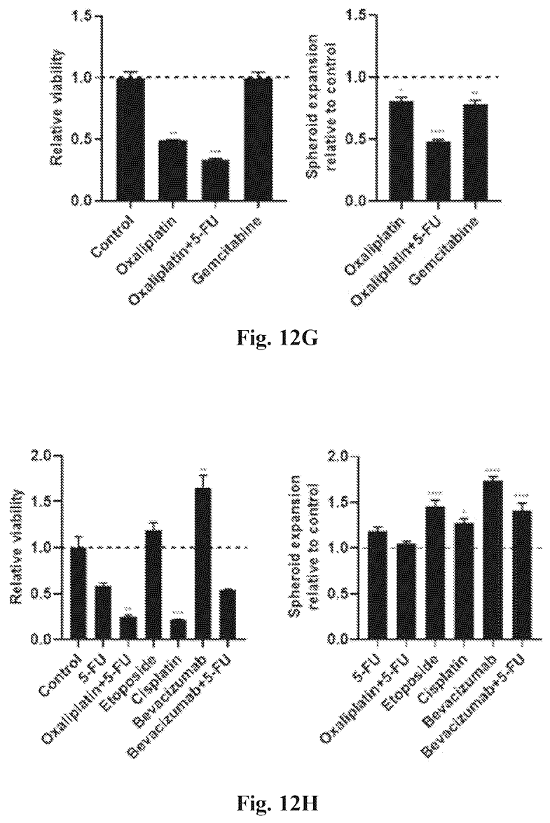

IC50 values for the various therapies are shown in FIGS. 11A-11F and Table 2. A Viability is displayed in FIGS. 12A-12I.

TABLE 2
IC50 values for various therapies

| | Cisplatin | Gemcitabine | 5-FU | Etoposide | Oxaliplatin | Mitomycin |
|---|---|---|---|---|---|---|
| AsPC-1 IC50 (μM) | 2.346 | 7.595E−1 | 1.357 | 4.107E−1 | 7.844 | 1.698 |
| BxPC-3 IC50 (μM) | 1.407 | 1.541E−4 | 1.212E−1 | 1.145E−2 | 7.605E−1 | 4.262E−1 |
| Panc1 IC50 (μM) | 12.47 | 6.923E−1 | 1.966 | 3.185 | 3.719 | 8.747 |

The viability of Oxaliplatin treated spheroids comprised of T1 cells, from a patient carrying a BRCA2 mutation, was reduced by ˜90%. Similar reduction in viability was observed with T4 and T5 spheroids, obtained from two patients carrying similar mutations (TP53, KRAS, PIK3K and SMAD4). Oxaliplatin induced a ˜56% reduction in viability, 5-FU a ˜45% reduction, while Gemcitabine induced only a mild reduction in viability.

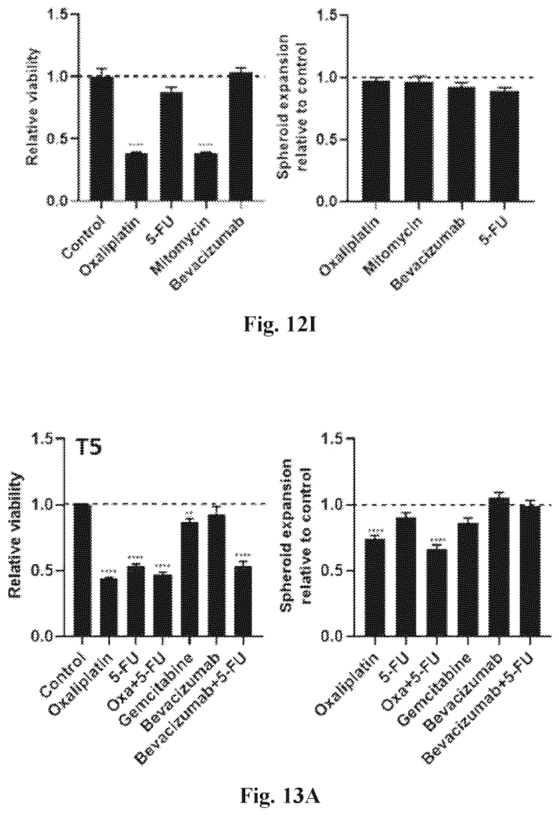

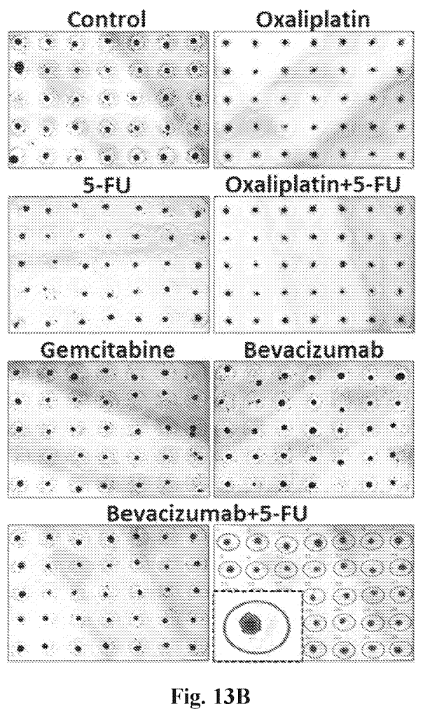

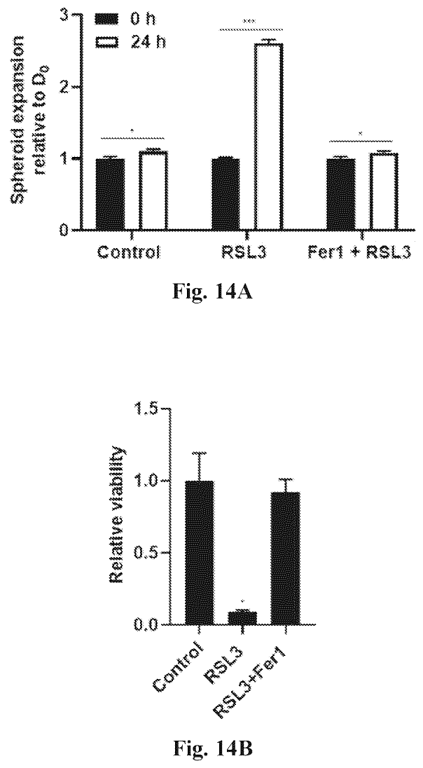

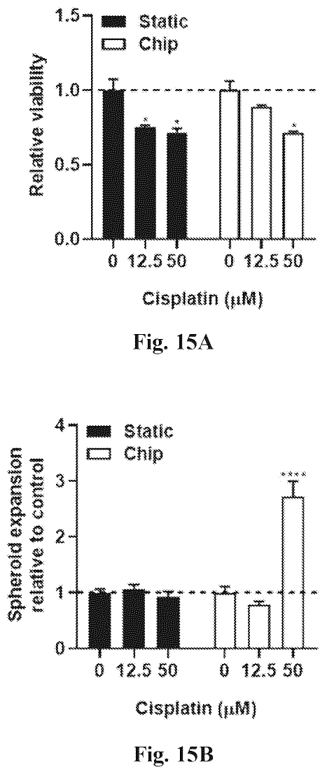

To study whether spheroid size corresponds to its viability over time, the size and viability were monitored via image analysis of brightfield top-view microscopical images and metabolism of tetrazolium salt, respectively. A direct trend was observed with spheroids treated with either Oxaliplatin, Gemcitabine, Etoposide or Mitomycin. T5 spheroids are an example showing a direct trend between reduction in viability and spheroid area over time under chemotherapy treatments (FIGS. 13A-13B). However, with 5-FU, Cisplatin and Bevacizumab the trend was frequently reversed. Similarly, a ferroptosis inducer, RSL3, brought about a significant reduction in BeWo spheroids' viability, while significantly increasing the spheroids' area (FIGS. 14A-14B). FIGS. 15A-15B display the viability and area change over time of BxPC-3 spheroids treated with Cisplatin at various concentrations under flow conditions versus static conditions. While the viability of spheroids was reduced in both flow and static conditions with increasing concentrations of Cisplatin, there is no significant change in the spheroids' area under static conditions, whereas in the flow conditions there is a significant increase in area (˜3-fold) with the highest Cisplatin concentration.

1. A method for obtaining a microfluidic device suitable for maintaining a biological sample in viable conditions, the method comprises:
(a) printing a first layer of a biocompatible UV-curable polymeric resin onto a substrate having a hydrophilic surface;
(b) applying UV radiation to cure said first layer;
(c) printing subsequent layers of said biocompatible UV-curable polymer resin onto the first layer, in a layer-by-layer mode according to pre-determined layer patterns, such that each of the subsequent layers having a thickness at least about an order of magnitude larger than that of the first layer, to form a microfluidics structure over said first layer;
(d) applying UV radiation to obtain a cured device;
(e) immersing said cured device in at least one solvent for a period of time sufficient to leach remainders of uncured biocompatible UV-curable polymeric resin from said cured device;
(f) removing said cured device from the solvent after said period of time to obtain said microfluidic device.

2. The method of claim 1, wherein the thickness of the first layer is at most about 0.05 mm.

3. The method of claim 1 or 2, wherein said biocompatible UV-curable polymeric resin is transparent.

4. The method of any one of claims 1 to 3, wherein said biocompatible UV-curable polymer resin comprises functionalized monomers selected from multifunctional epoxy and (meth)acrylate.

5. The method of any one of claims 1 to 4, wherein said first layer is substantially continuous over the hydrophilic surface.

6. The method of claim 5, wherein at least one of said subsequent layers is non-continuous to thereby form at least a portion of said microfluidics structure.

7. The method of any one of claims 1 to 6, wherein steps (a) and (c) are carried out in dark conditions.

8. The method of any one of claims 1 to 7, wherein said substrate is transparent.

9. The method of any one of claims 1 to 8, wherein said substrate is made of a hydrophilic polymer.

10. The method of any one of claims 1 to 9, wherein said substate is made of surface-treated plastic.

11. The method of any one of claims 1 to 10, wherein said substrate is made of glass, coated by one or more hydrophilic moieties.

12. The method of claim 11, comprising a step (0), before step (a), step (0) comprises coating a glass surface by one or more hydrophilic moieties.

13. The method of claim 12, wherein step (0) is carried out in dark conditions.

14. The method of any one of claims 1 to 13, wherein UV-radiation is applied between printing of each subsequent layer to at least partially cure said subsequent layer.

15. The method of any one of claims 1 to 14, wherein said solvent is at least one C₂-C₆ alcohol.

16. The method of any one of claims 1 to 15, wherein said period of time is at least about 6 hours.

17. A microfluidic device suitable for maintaining a biological sample in viable conditions obtained by the method of any one of claims 1 to 16.

18. A microfluidic device suitable for maintaining a biological sample in viable conditions, the microfluidic device comprising:
at least one fluid inlet port and at least one fluid outlet port defining a fluid flow path therebetween;
at least one biological sample holding chamber, positioned between the at least one fluid inlet port and at least one fluid outlet port in said flow path;
an array of fluid feed channels, the channels linking between said at least one inlet port and said at least one biological sample holding chamber in said flow path; and
at least one fluid draining channel linking said at least one biological sample holding chamber to said at least one outlet port in said flow path;
said microfluidic device being obtained by the method of any one of claims 1 to 16.

19. A microfluidic device suitable for maintaining one or more biological samples in viable conditions and exposing the biological samples to a plurality of different microenvironments, the microfluidic device comprising:
n fluid inlet ports and at least m fluid outlet ports defining a generally axial direction from the inlet ports to the outlet ports along a main plane of the device, the inlet ports and the outlet ports defining a fluid flow path therebetween;
m biological sample holding chambers, positioned between the fluid inlet ports and fluid outlet ports in said flow path;
an array of fluid feed channels linking between said inlet ports and said biological sample holding chambers in said flow path, the array comprises:
a distribution manifold,
at least one first set of at least m first channels, each first channel linking between the distribution manifold and a corresponding biological sample holding chamber, each of the first channels having at least a portion thereof defined in said main plane and at least one other portion thereof vertically distanced from said main plane; and
at plurality of fluid draining channels, corresponding to the number of biological sample holding chambers, each fluid draining channel linking between a biological sample holding chamber and a corresponding outlet port;
wherein n≥1, m is n+1.

20. The microfluidic device of claim 19, wherein n≥2.

21. The microfluidic device of claim 19 or 20, wherein the number of biological sample holding chambers is at least m+1, and the array comprises:
at least one second set of at least m+1 second fluid feed channels, each of the second channels having at least a portion thereof defined in said plane and at least one other portion thereof vertically distanced from said plane, and each second channel being linked to a corresponding biological sample holding chamber; and
a collection channel, linking between said first set and said second set.

22. A microfluidic device for maintaining one or more biological samples in viable conditions and exposing the biological samples to a plurality of different microenvironments, the microfluidic device comprising:
n fluid inlet ports and (n+i) fluid outlet ports defining a generally axial direction from the inlet ports to the outlet ports along a main plane of the device, the inlet ports and the outlet ports defining a fluid flow path therebetween;
(n+1) biological sample holding chambers, positioned between the fluid inlet ports and fluid outlet ports in said flow path;
an array of fluid feed channels linking between said inlet ports and said biological sample holding chambers in said flow path, the array comprises:
a distribution manifold,
p sets of fluid feed channels, the number of channels in each set p₍ᵢ₎ being n+i, each of fluid feed channels having at least a portion thereof defined in said main plane and at least one other portion thereof vertically distanced from said main plane; each first channel linking between the distribution manifold and a corresponding biological sample holding chamber;
(i−1) collection channels, each collection channel linking between two adjacent sets of fluid feed channels; and
(n+i) fluid draining channels, each fluid draining channel linking between one of the biological sample holding chambers and a corresponding outlet port;
wherein n is the number of inlet ports, n≥1,
p is the number of sets of fluid feed channels, p≥1, and
i is an integer index numeral counting the set of fluid feed channels, i≥1.

23. The microfluidic device of any one of claims 19 to 22, wherein the fluid feeding channels are curved.

24. The microfluidic device of any one of claims 19 to 23, wherein the fluid feeding channels are spiral.

25. The microfluidic device of any one of claims 19 to 24, wherein the transition between portions in each channel is via a channel segment that is perpendicular to the main plane.

26. The microfluidic device of any one of claims 19 to 25, being made of a transparent material.

27. The microfluidic device of any one of claims 19 to 26, being made of a biocompatible polymer.

28. The microfluidic device of any one of claims 19 to 27, wherein at least the inlet ports are configured to connect to fluid feed pumps.

29. The microfluidic device of any one of claims 19 to 28, wherein the inlet ports and outlet ports are configured to connect to a fluid feeding and collecting system.

30. The microfluidic device of any one of claims 19 to 29, obtainable by the method of any one of claims 1 to 16.

31. The microfluidic device of any one of claims 19 to 29, obtained by the method of any one of claims 1 to 16.

32. The microfluidic device of any one of claims 18 to 31, wherein:
the biological sample holding chambers are shaped as threaded cavities, and
the device comprises threaded caps, configured to be threadingly received in said threaded cavities.

33. The microfluidic device of claim 32, wherein said threaded caps are made of a transparent material.

34. The microfluidic device of any one of claims 18 to 31, comprising at least one biological sample introduction port, linked to the biological sample holding chambers, for introducing the biological sample into the chambers.

35. The microfluidic device of claim 34, comprising a plurality of biological sample introduction ports, corresponding to the number of biological sample holding chambers, each biological sample introduction ports being in fluid communication with a corresponding biological sample holding chamber.

36. The microfluidic device of claim 34 or 35, wherein the biological sample introduction port(s) are configured to be linkable to a biological sample reservoir.

37. The microfluidic device of claim 34 or 35, wherein the biological sample introduction port(s) are configured to be linkable to a hanging drop unit.

38. A microfluidic device suitable for maintaining a biological sample in viable conditions, the microfluidic device comprising:
at least one fluid inlet port and at least one fluid outlet port defining a fluid flow path therebetween;
at least one biological sample holding chamber shaped as a threaded cavity, and positioned between the at least one fluid inlet port and at least one fluid outlet port in said flow path;
at least one corresponding threaded cap, configured to be threadingly received in said threaded cavity;
an array of fluid feed channels, the channels linking between said at least one inlet port and said at least one biological sample holding chamber in said flow path; and
at least one fluid draining channel linking said at least one biological sample holding chamber to said at least one outlet port in said flow path.

39. The microfluidic device of claim 38, made of a transparent material.

40. The microfluidic device of claim 38 or 39, being made of a biocompatible polymer.

41. The microfluidic device of any one of claims 38 to 40, wherein the threaded cap is made of a transparent material.

42. The microfluidic device of any one of claims 38 to 41, obtained by the method of any one of claims 1 to 16.

43. A kit comprising:
a microfluidic device suitable for maintaining a biological sample in viable conditions, the microfluidic device comprising at least one fluid inlet port and at least one fluid outlet port defining a fluid flow path therebetween; at least one biological sample holding chamber shaped as a threaded cavity, and positioned between the at least one fluid inlet port and at least one fluid outlet port in said flow path; an array of fluid feed channels, the channels linking between said at least one inlet port and said at least one biological sample holding chamber in said flow path; and at least one fluid draining channel linking said at least one biological sample holding chamber to said at least one outlet port in said flow path; and
at least one corresponding threaded cap, configured to be threadingly received in said threaded cavity.

44. A method of determining a biological sample response to an environment ex vivo, the method comprising:
introducing the biological sample into a biological sample holding chamber of a microfluidic device of any one of claims 18 to 42;
introducing one or more fluids into the flow path of the microfluidic device through the inlet port(s) to expose said biological sample to a desired environment; and
analyzing the response of said biological sample to said environment.

45. The method of claim 44, wherein the microfluidic device comprises at least two ports, and the method comprises introducing a different fluid through each port.
